(19) United States
(12) Patent Application Publication
(10) Pub. No.: US 2006/0204493 A1
(43) Pub. Date: Sep. 14, 2006

(54) Heteromultimeric molecules

(75) Inventors: Arthur J. Huang, Oakland, CA (US); Barbara Moffat, Berkeley, CA (US); Daniel G. Yansura, Pacifica, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 11/218,286

(22) Filed: Sep. 1, 2005

Related U.S. Application Data
(60) Provisional application No. 60/607,172, filed on Sep. 2, 2004.

(51) Int. Cl. A61K 39/395; C07H 21/04; C12P 21/06; C07K 16/44; C12N 5/06

(52) U.S. Cl. 424/133.1; 435/69.1; 435/320.1; 435/328; 530/387.3; 536/23.53

(57) ABSTRACT

The invention provides heteromultimeric antibodies, and methods of making these antibodies at high yields and purity. The invention also provides methods and compositions for using these antibodies.

### RELATED APPLICATIONS

This application is a non-provisional application filed under 37 CFR 1.53(b)(1), claiming priority under 35 USC 119(e) to provisional application No. 60/607,172 filed Sep. 2, 2004, the contents of which are incorporated herein in their entirety by reference.

### FIELD OF THE INVENTION

This invention relates to a method for making heteromultimeric polypeptides such as multispecific antibodies (e.g. bispecific antibodies), multispecific immunoadhesins (e.g. bispecific immunoadhesins) as well as antibody-immunoadhesin chimeras and the heteromultimeric polypeptides made using the method.

### BACKGROUND

Bispecific Antibodies

Bispecific antibodies (BsAbs) which have binding specificities for at least two different antigens have significant potential in a wide range of clinical applications as targeting agents for in vitro and in vivo immunodiagnosis and therapy, and for diagnostic immunoassays. See, generally, Segal et al., J. Immunol. Methods (2001), 248:1-6; Kufer et al., Trends in Biotech. (2004), 22(5):238-244; van Spriel et al., Immunol. Today (2000), 21(8):391-397; Talac & Nelson, J. Biol. Reg. & Homeostatic Agents (2000), 14(3):175-181; Hayden et al., Curr. Op. Immunol. (1997), 9:201-212; Carter, J. Immunol. Methods (2001), 248:7-15; Peipp & Valerius, Biochem. Soc. Trans. (2002), 30(4):507-511; Milstein & Cuello, Nature (1983), 305:537-540; Karpovsky et al., J. Exp. Med. (1984), 160:1686-1701; Perez et al., Nature (1985), 316:354-356; Canevari et al., J. Natl. Cancer Inst. (1995), 87:1463-1469; Kroesen et al., Br. J. Cancer (1994), 70:652-661; Valone et al., J. Clin. Oncol. (1995), 13:2281-2292; Weiner et al., Cancer Res. (1995), 55:4586-4593; Muller et al., FEBS Letters (1998), 422:259-264.

In the diagnostic areas, bispecific antibodies have been very useful in probing the functional properties of cell surface molecules and in defining the ability of the different Fc receptors to mediate cytotoxicity (Fanger et al., Crit. Rev. Immunol. 12:101-124 [1992]). Nolan et al., Biochem. Biophys. Acta. 1040:1-11 (1990) describe other diagnostic applications for BsAbs. In particular, BsAbs can be constructed to immobilize enzymes for use in enzyme immunoassays. To achieve this, one arm of the BsAb can be designed to bind to a specific epitope on the enzyme so that binding does not cause enzyme inhibition, the other arm of the BsAb binds to the immobilizing matrix ensuring a high enzyme density at the desired site. Examples of such diagnostic BsAbs include the rabbit anti-IgG/anti-ferritin BsAb described by Hammerling et al., J. Exp. Med. 128:1461-1473 (1968) which was used to locate surface antigens. BsAbs having binding specificities for horse radish peroxidase (HRP) as well as a hormone have also been developed. Another potential immunochemical application for BsAbs involves their use in two-site immunoassays. For example, two BsAbs are produced binding to two separate epitopes on the analyte protein—one BsAb binds the complex to an insoluble matrix, the other binds an indicator enzyme (see Nolan et al., supra).

Bispecific antibodies can also be used for in vitro or in vivo immunodiagnosis of various diseases such as cancer (Songsivilai et al., Clin. Exp. Immunol. 79:315 [1990]). To facilitate this diagnostic use of the BsAb, one arm of the BsAb can bind a tumor associated antigen and the other arm can bind a detectable marker such as a chelator which tightly binds a radionuclide. Using this approach, Le Doussal et al. made a BsAb useful for radioimmunodetection of colorectal and thryoid carcinomas which had one arm which bound a carcinoembryonic antigen (CEA) and another arm which bound diethylenetriaminepentacetic acid (DPTA). See Le Doussal et al., Int. J. Cancer Suppl. 7:58-62 (1992) and Le Doussal et al., J. Nucl. Med. 34:1662-1671 (1993). Stickney et al. similarly describe a strategy for detecting colorectal cancers expressing CEA using radioimmunodetection. These investigators describe a BsAb which binds CEA as well as hydroxyethylthiourea-benzyl-EDTA (EOTUBE). See Stickney et al., Cancer Res. 51:6650-6655 (1991).

Bispecific antibodies can also be used for human therapy, for example in redirected cytotoxicity by providing one arm which binds a target (e.g. pathogen or tumor cell) and another arm which binds a cytotoxic trigger molecule, such as the T-cell receptor or the Fcγ receptor. Accordingly, bispecific antibodies can be used to direct a patient's cellular immune defense mechanisms specifically to the tumor cell or infectious agent. Using this strategy, it has been demonstrated that bispecific antibodies which bind to the FcγRIII (i.e. CD16) can mediate tumor cell killing by natural killer (NK) cell/large granular lymphocyte (LGL) cells in vitro and are effective in preventing tumor growth in vivo. Segal et al., Chem. Immunol. 47:179 (1989) and Segal et al., Biologic Therapy of Cancer 2(4) DeVita et al. eds. J. B. Lippincott, Philadelphia (1992) p. 1. Similarly, a bispecific antibody having one arm which binds FcγRIII and another which binds to the HER2 receptor has been developed for therapy of ovarian and breast tumors that overexpress the HER2 antigen. (Hseih-Ma et al. Cancer Research 52:6832-6839 [1992] and Weiner et al. Cancer Research 53:94-100 [1993]). Bispecific antibodies can also mediate killing by T cells. Normally, the bispecific antibodies link the CD3 complex on T cells to a tumor-associated antigen. A fully humanized F(ab′)₂ BsAb consisting of anti-CD3 linked to anti-p185^{HER2} has been used to target T cells to kill tumor cells overexpressing the HER2 receptor. Shalaby et al., J. Exp. Med. 175(1):217 (1992). Bispecific antibodies have been tested in several early phase clinical trials with encouraging results. In one trial, 12 patients with lung, ovarian or breast cancer were treated with infusions of activated T-lymphocytes targeted with an anti-CD3/anti-tumor (MOC31) bispecific antibody. deLeij et al. Bispecific Antibodies and Targeted Cellular Cytotoxicity, Romet-Lemonne, Fanger and Segal Eds., Lienhart (1991) p. 249. The targeted cells induced considerable local lysis of tumor cells, a mild inflammatory reaction, but no toxic side effects or anti-mouse antibody responses. In a very preliminary trial of an anti-CD3/anti-CD19 bispecific antibody in a patient with B-cell malignancy, significant reduction in peripheral tumor cell counts was also achieved. Clark et al. Bispecific Antibodies and Targeted Cellular Cytotoxicity, Romet-Lemonne, Fanger and Segal Eds., Lienhart (1991) p. 243. See also Kroesen et al., Cancer Immunol. Immunother. 37:400-407 (1993), Kroesen et al., Br. J. Cancer 70:652-661 (1994) and Weiner et al., J. Immunol. 152:2385 (1994) concerning therapeutic applications for BsAbs.

Bispecific antibodies may also be used as fibrinolytic agents or vaccine adjuvants. Furthermore, these antibodies may be used in the treatment of infectious diseases (e.g. for targeting of effector cells to virally infected cells such as HIV or influenza virus or protozoa such as Toxoplasma gondii), used to deliver immunotoxins to tumor cells, or target immune complexes to cell surface receptors (see Fanger et al., supra).

Use of BsAbs has been effectively stymied by the difficulty of obtaining BsAbs in sufficient quantity and purity. Traditionally, bispecific antibodies were made using hybrid-hybridoma technology (Millstein and Cuello, Nature 305:537-539 [1983]). Because of the random assortment of immunoglobulin heavy and light chains, these hybridomas (quadromas) produce a potential mixture of 10 different antibody molecules, of which only one has the correct bispecific structure. Accordingly, techniques for the production of greater yields of BsAb have been attempted. For example, bispecific antibodies can be prepared using chemical linkage. To achieve chemical coupling of antibody fragments, Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the BsAb. The BsAbs produced can be used as agents for the selective immobilization of enzymes.

Recent progress has facilitated the direct recovery of Fab′-SH fragments from E. coli. which can be chemically coupled to form bispecific antibodies. Shalaby et al., J. Exp. Med. 175:217-225 (1992) describe the production of a fully humanized BsAb F(ab′)₂ molecule having one arm which binds p185^{HER2} and another arm which binds CD3. Each Fab′ fragment was separately secreted from E. coli. and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also Rodrigues et al., Int. J. Cancers (Suppl.) 7:45-50 (1992).

However, options for producing bispecific antibodies that are larger than Fab or Fab′ fragments generally remain scarce. Moreover, in many instances, the use of chemical coupling in vitro present undesirable problems.

Various techniques for making and isolating BsAb fragments directly from recombinant cell cultures have also been described. For example, bispecific F(ab′)₂ heterodimers have been produced using leucine zippers. Kostelny et al., J. Immunol. 148(5): 1547-1553 (1992). The leucine zipper peptides from the Fos and Jun proteins were linked to the Fab′ portions of anti-CD3 and anti-interleukin-2 receptor (IL-2R) antibodies by gene fusion. The antibody homodimers were reduced at the hinge region to form monomers and then reoxidized to form the antibody heterodimers. The BsAbs were found to be highly effective in recruiting cytotoxic T cells to lyse HuT-102 cells in vitro. The advent of the “diabody” technology described by Hollinger et al., PNAS (USA) 90:6444-6448 (1993) has provided an alternative mechanism for making BsAb fragments. The fragments comprise a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making BsAb fragments by the use of single chain Fv (sFv) dimers has also been reported. See Gruber et al. J. Immunol. 152:5368 (1994). These researchers designed an antibody which comprised the V_{H} and V_{L} domains of an antibody directed against the T cell receptor joined by a 25 amino acid residue linker to the V_{H} and V_{L} domains of an anti-fluorescein antibody. The refolded molecule bound to fluorescein and the T cell receptor and redirected the lysis of human tumor cells that had fluorescein covalently linked to their surface.

It is apparent that several techniques for making bispecific antibody fragments which can be recovered directly from recombinant cell culture have been reported. However, full or substantially full length BsAbs may be preferable to BsAb fragments for many clinical applications because of their likely longer serum half-life and possible effector functions. An elegant method reported to be useful for making such BsAbs is described in U.S. Pat. Nos. 5,731,168; 5,821,333; and 5,807,706; and Merchant et al., Nat. Biotech. (1998), 16:677-681, although the method primarily provides for generating bispecific antibodies having a common light chain, and requires separating out any excess monospecific antibody to obtain substantially pure preparations of a desired bispecific antibody.

Immunoadhesins

Immunoadhesins (Ia's) are antibody-like molecules which combine the binding domain of a protein such as a cell-surface receptor or a ligand (an “adhesin”) with the effector functions of an immunoglobulin constant domain. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use.

Immunoadhesins reported in the literature include fusions of the T cell receptor (Gascoigne et al., Proc. Natl. Acad. Sci. USA 84:2936-2940 [1987]); CD4 (Capon et al., Nature 337:525-531 [1989]; Traunecker et al., Nature 339:68-70 [1989]; Zettmeissl et al., DNA Cell Biol. USA 9:347-353 [1990]; and Byrn et al., Nature 344:667-670 [1990]); L-selectin or homing receptor (Watson et al., J. Cell. Biol. 110:2221-2229 [1990]; and Watson et al., Nature 349:164-167 [1991]); CD44 (Aruffo et al., Cell 61:1303-1313 [1990]); CD28 and B7 (Linsley et al., J. Exp. Med. 173:721-730 [1991]); CTLA-4 (Lisley et al., J. Exp. Med. 174:561-569 [1991]); CD22 (Stamenkovic et al., Cell 66:1133-1144 [1991]); TNF receptor (Ashkenazi et al., Proc. Natl. Acad. Sci. USA 88:10535-10539 [1991]; Lesslauer et al., Eur. J. Immunol. 27:2883-2886 [1991]; and Peppel et al., J. Exp. Med. 174:1483-1489 [1991]); NP receptors (Bennett et al., J. Biol. Chem. 266:23060-23067 [1991]); inteferon γ receptor (Kurschner et al., J. Biol. Chem. 267:9354-9360 [1992]); 4- 1BB (Chalupny et al., PNAS [USA] 89:10360-10364 [1992]) and IgE receptor a (Ridgway and Gorman, J. Cell. Biol. Vol. 115, Abstract No. 1448 [1991]).

Examples of immunoadhesins which have been described for therapeutic use include the CD4-IgG immunoadhesin for blocking the binding of HIV to cell-surface CD4. Data obtained from Phase I clinical trials in which CD4-IgG was administered to pregnant women just before delivery suggests that this immunoadhesin may be useful in the prevention of maternal-fetal transfer of HIV. Ashkenazi et al., Intern. Rev. Immunol. 10:219-227 (1993). An immunoadhesin which binds tumor necrosis factor (TNF) has also been developed. TNF is a proinflammatory cytokine which has been shown to be a major mediator of septic shock. Based on a mouse model of septic shock, a TNF receptor immunoadhesin has shown promise as a candidate for clinical use in treating septic shock (Ashkenazi et al., supra). Immunoadhesins also have non-therapeutic uses. For example, the L-selectin receptor immunoadhesin was used as an reagent for histochemical staining of peripheral lymph node high endothelial venules (HEV). This reagent was also used to isolate and characterize the L-selectin ligand (Ashkenazi et al., supra).

If the two arms of the immunoadhesin structure have different specificities, the immunoadhesin is called a “bispecific immunoadhesin” by analogy to bispecific antibodies. Dietsch et al., J. Immunol. Methods 162:123 (1993) describe such a bispecific immunoadhesin combining the extracellular domains of the adhesion molecules, E-selectin and P-selectin. Binding studies indicated that the bispecific immunoglobulin fusion protein so formed had an enhanced ability to bind to a myeloid cell line compared to the monospecific immunoadhesins from which it was derived.

Antibody-Immunoadhesin Chimeras

Antibody-immunoadhesin (Ab/Ia) chimeras have also been described in the literature. These molecules combine the binding region of an immunoadhesin with the binding domain of an antibody.

Berg et al., PNAS (USA) 88:4723-4727 (1991) made a bispecific antibody-immunoadhesin chimera which was derived from murine CD4-IgG. These workers constructed a tetrameric molecule having two arms. One arm was composed of CD4 fused with an antibody heavy-chain constant domain along with a CD4 fusion with an antibody light-chain constant domain. The other arm was composed of a complete heavy-chain of an anti-CD3 antibody along with a complete light-chain of the same antibody. By virtue of the CD4-IgG arm, this bispecific molecule binds to CD3 on the surface of cytotoxic T cells. The juxtaposition of the cytotoxic cells and HIV-infected cells results in specific killing of the latter cells.

While Berg et al. describe a bispecific molecule that was tetrameric in structure, it is possible to produce a trimeric hybrid molecule that contains only one CD4-IgG fusion. See Chamow et al., J. Immunol. 153:4268 (1994). The first arm of this construct is formed by a humanized anti-CD3 κ light chain and a humanized anti-CD3 γ heavy chain. The second arm is a CD4-IgG immunoadhesin which combines part of the extracellular domain of CD4 responsible for gp120 binding with the Fc domain of IgG. The resultant Ab/Ia chimera mediated killing of HIV-infected cells using either pure cytotoxic T cell preparations or whole peripheral blood lymphocyte (PBL) fractions that additionally included Fc receptor-bearing large granular lymphocyte effector cells.

In the manufacture of the above-mentioned heteromultimers, it is desirable to increase the yields of the desired heteromultimer over the homomultimer(s), in particular full or substantially ful length heteromultimeric molecules at significant purity. The invention described herein provides a means for achieving this.

All references cited herein, including patent applications and publications, are incorporated by reference in their entirety.

### DISCLOSURE OF THE INVENTION

The invention provides methods of producing antibodies capable of specifically binding to more than one target (e.g., epitopes on a single molecule or on different molecules). The invention also provides methods of using these antibodies, and compositions, kits and articles of manufacture comprising these antibodies.

The invention provides efficient and novel methods of producing multispecific immunoglobulin complexes (e.g., bispecific antibodies) that overcome limitations of traditional methods. Multispecific immunoglobulin complexes, such as bispecific antibodies, can be provided as a highly homogeneous heteromultimer polypeptide according to methods of the invention.

In one aspect, the invention provides a method of making an antibody comprising a first heavy chain polypeptide paired with a first light chain polypeptide, and a second heavy chain polypeptide paired with a second light chain polypeptide, wherein the first heavy chain polypeptide and the second heavy chain polypeptide each comprises a variant hinge region incapable of inter-heavy chain disulfide linkage, said method comprising:

(a) expressing the first heavy chain polypeptide and the first light chain polypeptide in a first host cell;

(b) expressing the second heavy chain polypeptide and the second light chain polypeptide in a second host cell;

(c) isolating the heavy and light chain polypeptides of (a) and (b);

(d) annealing (or combining or contacting) the isolated polypeptides of (c) to form a multispecific antibody comprising a first arm comprising the first heavy chain paired with the first light chain, and a second arm comprising the second heavy chain paired with the second light chain.

In one aspect, the invention provides a method of making a multispecific immunoglobulin complex comprising a first target binding polypeptide and a second target binding polypeptide, wherein the first polypeptide and the second polypeptide each comprises a variant heavy chain hinge region incapable of inter-heavy chain disulfide linkage, said method comprising:

(a) expressing the first polypeptide in a first host cell;

(b) expressing the second polypeptide in a second host cell;

(c) isolating the polypeptides of (a) and (b);

(d) annealing (or combining or contacting) the isolated polypeptides of (c) to form a multispecific immunoglobulin complex comprising a first target binding polypeptide and a second target binding polypeptide.

In one aspect, the invention provides a method comprising:

(a) expressing in a first host cell a first pair of immunoglobulin heavy and light chain polypeptides that are capable of forming a first target molecule binding arm,

(b) expressing in a second host cell a second pair of immunoglobulin heavy and light chain polypeptides that are capable of forming a second target molecule binding arm,

wherein heavy chain polypeptides of the first pair and second pair each comprises a variant hinge region incapable of inter-heavy chain disulfide linkage, and wherein light chains of the first pair and second pair comprise different antigen binding determinants (e.g., different variable domain sequences),

(c) isolating the polypeptides from the host cells of steps (a) and (b),

(d) contacting the polypeptides in vitro under conditions permitting multimerization of the isolated polypeptides to form a substantially homogeneous population of antibodies having binding specificity to at least two distinct target molecules.

In one aspect, the invention provides a method comprising:

(a) expressing in a first host cell a first polypeptide that is capable of forming a first target molecule binding entity,

(b) expressing in a second host cell a second polypeptide that is capable of forming a second target molecule binding entity,

wherein the first and second polypeptide each comprises an Fc sequence/region (e.g., a variant heavy chain hinge region as described herein) incapable of inter-heavy chain disulfide linkage, and wherein the first and second polypeptide comprise different antigen binding determinants (e.g., different variable domain sequences),

(c) isolating the polypeptides from the host cells of steps (a) and (b),

(d) contacting the polypeptides in vitro under conditions permitting multimerization of the isolated polypeptides to form a substantially homogeneous population of multimeric polypeptides, wherein each multimer has binding specificity to at least two distinct target molecules.

In one aspect, the invention provides a method comprising:

(a) obtaining a sample comprising a mixture of at least 4 different polypeptides, wherein the 4 polypeptides are a first pair of immunoglobulin heavy and light chain polypeptides that are capable of forming a first target molecule binding arm, and a second pair of immunoglobulin heavy and light chain polypeptides that are capable of forming a second target molecule binding arm, wherein heavy chain polypeptides of the first pair and second pair each comprises a variant hinge region incapable of inter-heavy chain disulfide linkage,

(b) incubating the 4 polypeptides under conditions permitting multimerization of the polypeptides to form a substantially homogeneous population of antibodies having binding specificity to at least two distinct target molecules.

In one aspect, the invention provides a method comprising:

incubating at least 4 immunoglobulin polypeptides under conditions permitting multimerization of the polypeptides to form a substantially homogeneous population of antibodies, wherein each antibody has binding specificity to at least two distinct target molecules,

wherein the 4 immunoglobulin polypeptides are a first pair of immunoglobulin heavy and light chain polypeptides that are capable of forming a first target molecule binding arm, and a second pair of immunoglobulin heavy and light chain polypeptides that are capable of forming a second target molecule binding arm,

wherein each heavy chain polypeptide of the first pair and second pair comprises a variant hinge region incapable of inter-heavy chain disulfide linkage.

In one aspect, the invention provides a method comprising:

(a) obtaining a sample comprising at least 2 polypeptides, wherein at least one polypeptide is capable of forming a first target molecule binding arm, and at least one polypeptide is capable of forming a second target molecule binding arm, wherein the first target molecule binding arm and the second target molecule binding arm each comprises an immunoglobulin heavy chain variant hinge region incapable of inter-heavy chain disulfide linkage,

(b) incubating the polypeptides under conditions permitting multimerization of the polypeptides to form a substantially homogeneous population of multimeric polypeptides, wherein each multimer has binding specificity to at least two distinct target molecules.

In one aspect, the invention provides a method comprising:

incubating at least 4 immunoglobulin polypeptides under conditions permitting multimerization of the polypeptides to form a substantially homogeneous population of antibodies, wherein each antibody has binding specificity to at least two distinct target molecules,

wherein the 4 immunoglobulin polypeptides are a first pair of immunoglobulin heavy and light chain polypeptides that are capable of forming a first target molecule binding arm, and a second pair of immunoglobulin heavy and light chain polypeptides that are capable of forming a second target molecule binding arm,

wherein each heavy chain polypeptide of the first pair and second pair comprises a variant hinge region incapable of inter-heavy chain disulfide linkage.

In one aspect, the invention provides a method comprising:

incubating at least 4 immunoglobulin polypeptides under conditions permitting multimerization of the polypeptides to form a substantially homogeneous population of multimeric polypeptides, wherein each multimer has binding specificity to at least two distinct target molecules,

wherein the at least 4 immunoglobulin polypeptides form a first pair of polypeptides that are capable of forming a fist target molecule binding arm, and a second pair of polypeptides that are capable of forming a second target molecule binding arm,

wherein the first target molecule binding arm and the second target molecule binding arm each comprises a variant immunoglobulin heavy chain hinge region incapable of inter-heavy chain disulfide linkage.

In one aspect, the invention provides a method comprising:

incubating a first pair of immunoglobulin heavy and light chain polypeptides, and a second pair of immunoglobulin heavy and light chain polypeptides, under conditions permitting multimerization of the first and second pair of polypeptides to form a substantially homogeneous population of antibodies,

wherein the first pair of polypeptides is capable of binding a first target molecule;

wherein the second pair of polypeptides is capable of binding a second target molecule;

wherein each heavy chain polypeptide of the first pair and second pair comprises a variant hinge region incapable of inter-heavy chain disulfide linkage.

In one aspect, the invention provides a method comprising:

incubating a first polypeptide complex, and a second polypeptide complex, under conditions permitting multimerization of the first and second polypeptide complex to form a substantially homogeneous population of multimeric polypeptides, wherein each multimer has binding specificity to at least two distinct target molecules,

wherein the first polypeptide complex is capable of binding a first target molecule;

wherein the second polypeptide complex is capable of binding a second target molecule;

wherein each polypeptide complex comprises a variant immunoglobulin heavy chain hinge region incapable of inter-heavy chain disulfide linkage.

In one aspect, the invention provides a method comprising:

incubating a first pair of immunoglobulin heavy and light chain polypeptides, and a second pair of immunoglobulin heavy and light chain polypeptides, under in vitro conditions permitting multimerization of the first and second pair of polypeptides to form a substantially homogeneous population of antibodies,

wherein the first pair of polypeptides is capable of binding a first target molecule;

wherein the second pair of polypeptides is capable of binding a second target molecule;

wherein Fc polypeptide of the first heavy chain polypeptide and Fc polypeptide of the second heavy chain polypeptide meet at an interface, and the interface of the second Fc polypeptide comprises a protuberance which is positionable in a cavity in the interface of the first Fc polypeptide.

In one aspect, the invention provides a method comprising:

incubating a first polypeptide and a second polypeptide under in vitro conditions permitting multimerization of the first and second polypeptide to form a substantially homogeneous population of multimers, wherein each polypeptide comprises at least a portion (including all) of an immunoglobulin heavy chain Fc region (e.g., CH2 and/or CH3), wherein each multimer is capable of binding to at least two distinct target molecules,

wherein the first polypeptide is capable of binding a first target molecule;

wherein the second polypeptide is capable of binding a second target molecule;

wherein Fc sequence the first polypeptide and Fc sequence of the second polypeptide meet at an interface, and the interface of the second Fc sequence comprises a protuberance which is positionable in a cavity in the interface of the first Fc sequence.

In some embodiments of methods of the invention, the multispecific antibody that is generated comprises a variant heavy chain hinge region that lacks at least one of the inter-heavy chain disulfide linkages normally present in wild type full length antibodies. For example, in one embodiment, methods of the invention provide a bispecific antibody in which at least one inter-heavy chain disulfide linkage is eliminated. In some embodiments, said antibody is one in which at least two, or any interger number up to all inter-heavy chain disulfide linkages are eliminated. In some embodiments, said antibody is one in which all inter-heavy chain disulfide linkages are eliminated. Thus, in some embodiments, said antibody comprises a variant heavy chain incapable of inter-heavy chain disulfide linkage. In one embodiment, said antibody comprises a variant heavy chain hinge region varied such that at least one inter-heavy chain disulfide linkage is eliminated. In one embodiment, said antibodies comprise a variant immunoglobulin hinge region that lacks at least one, at least two, at least three, at least four, or any interger number up to all, of the cysteine residues that are normally capable of forming an inter-heavy chain disulfide linkage. A variant hinge region can be rendered lacking in said cysteine residue(s) by any suitable method including deletion, substitution or modification of said residue(s). In one embodiment, said cysteine(s) is one that is normally capable of intermolecular disulfide linkage, e.g. between cysteines of two immunoglobulin heavy chains. In some embodiments of these methods, all inter-heavy chain disulfide linkage-forming hinge cysteines of the variant heavy chain are rendered incapable of forming a disulfide linkage.

Any of a number of host cells can be used in methods of the invention. Such cells are known in the art (some of which are described herein) or can be determined empricially with respect to suitability for use in methods of the invention using routine techniques known in the art. In one embodiment, a host cell is prokaryotic. In some embodiments, a host cell is a gram-negative bacterial cell. In one embodiment, a host cell is E. coli. In some embodiments, the E. coli is of a strain deficient in endogenous protease activities. In some embodiments, the genotype of an E. coli host cell lacks degP and prc genes and harbors a mutant spr gene. In one embodiment, a host cell is mammalian, for example, a Chinese Hamster Ovary (CHO) cell.

In some embodiments, methods of the invention further comprise expressing in a host cell a polynucleotide or recombinant vector encoding a molecule the expression of which in the host cell enhances yield of an antibody of the invention. For example, such molecule can be a chaperone protein. In one embodiment, said molecule is a prokaryotic polypeptide selected from the group consisting of DsbA, DsbC, DsbG and FkpA. In some embodiments of these methods, the polynucleotide encodes both DsbA and DsbC.

Antibodies expressed in prokaryotic cells such as E. coli are aglycosylated. Thus, in some aspects, the invention provides aglycosylated multispecific antibodies obtained according to methods of the invention.

Antibodies expressed in host cells according to methods of the invention can be recovered from the appropriate cell compartment or medium. Factors that determine route of antibody recovery are known in the art, including, for example, whether a secretion signal is present on the antibody polypeptide, culture conditions, host genetic background (for example, some hosts can be made to leak proteins to the supernatant), etc. In some embodiments, antibody generated according to methods of the invention is recovered from cell lysate. In some embodiments, antibody generated according to methods of the invention is recovered from the periplasm or culture medium.

In one aspect, the invention provides a multispecific antibody lacking inter-heavy chain disulfide linkage. In some embodiments, said inter-heavy chain disulfide linkage is between Fc regions. In another aspect, the invention provides multispecific antibodies comprising a variant heavy chain hinge region incapable of inter-heavy chain disulfide linkage. In one embodiment, said variant hinge region lacks at least one cysteine, at least two, at least three, at least four or preferably any interger number up to all cysteines capable of forming an inter-heavy chain disulfide linkage.

Antibodies of the invention are useful for various applications and in a variety of settings. Preferably, antibodies of the invention are biologically active. Preferably, antibodies of the invention possess substantially similar biological characteristics (such as, but not limited to, antigen binding capability) and/or physicochemical characteristics as their wild type counterparts (i.e., antibodies that differ from the antibodies of the invention primarily or solely with respect to the extent they are capable of disulfide linkage formation, e.g., as determined by whether one or more hinge cysteines is rendered incapable of disulfide linkage formation).

In antibodies and methods of the invention, a cysteine residue can be rendered incapable of forming a disulfide linkage by any of a number of methods and techniques known in the art. For example, a hinge region cysteine that is normally capable of forming a disulfide linkage may be deleted. In another example, a cysteine residue of the hinge region that is normally capable of forming a disulfide linkage may be substituted with another amino acid, such as, fcr example, serine. In some embodiments, a hinge region cysteine residue may be modified such that it is incapable of disulfide bonding.

Antibodies of the invention can be of any of a variety of forms. In one embodiment, an antibody of the invention is a full-length antibody or is substantially full length (i.e., comprises a complete or almost complete heavy chain sequence, and a complete or almost complete light chain sequence). In one aspect, the invention provides an antibody that is humanized. In another aspect, the invention provides a human antibody. In another aspect, the invention provides a chimeric antibody.

An antibody of the invention may also be an antibody fragment, such as, for example, an Fc or Fc fusion polypeptide. An Fc fusion polypeptide generally comprises an Fc sequence (or fragment thereof) fused to a heterologous polypeptide sequence (such as an antigen binding domain), such as a receptor extracellular domain (ECD) fused to an immunoglobulin Fc sequence (e.g., Fit receptor ECD fused to a IgG2 Fc). For example, in one embodiment, an Fc fusion polypeptide comprises a VEGF binding domain, which may be a VEGF receptor, which includes flt, flk, etc. An antibody of the invention generally comprises a heavy chain constant domain and a light chain constant domain. In some embodiments, an antibody of the invention does not contain an added, substituted or modified amino acid in the Fc region, preferably the hinge region, that is capable of inter-heavy chain disulfide linkage. In one embodiment, an antibody of the invention does not comprise a modification (for example, but not limited to, insertion of one or more amino acids, e.g., to form a dimerization sequence such as leucine zipper) for formation of inter-heavy chain dimerization or multimerization. In some embodiments, a portion (but not all) of the Fc sequence is missing in an antibody of the invention. In some of these embodiments, the missing Fc sequence is a portion or all of the CH2 and/or CH3 domain. In some of these embodiments, the antibody comprises a dimerization domain (such as a leucine zipper sequence), for example fused to the C-terminus of the heavy chain fragment.

In some embodiments of methods and antibodies of the invention, the heavy chain polypeptides comprise at least one characteristic that promotes heterodimerization, while minimizing homodimerization, of the first and second heavy chain polypeptides (i.e., between Fc sequences of the heavy chains). Such characteristic(s) improves yield and/or purity and/or homogeneity of the immunoglobulin populations obtainable by methods of the invention as described herein. In one embodiment, Fc sequence of a first heavy chain polypeptide and a second heavy chain polypeptide meet/interact at an interface. In some embodiments wherein Fc sequence of the first and second Fc polypeptides meet at an interface, the interface of the second Fc polypeptide (sequence) comprises a protuberance which is positionable in a cavity in the interface of the first Fc polypeptide (sequence). In one embodiment, the first Fc polypeptide has been altered from a template/original polypeptide to encode the cavity or the second Fc polypeptide has been altered from a template/original polypeptide to encode the protuberance, or both. In one embodiment, the first Fc polypeptide has been altered from a template/original polypeptide to encode the cavity and the second Fc polypeptide has been altered from a template/original polypeptide to encode the protuberance, or both. In one embodiment, the interface of the second Fc polypeptide comprises a protuberance which is positionable in a cavity in the interface of the first Fc polypeptide, wherein the cavity or protuberance, or both, have been introduced into the interface of the first and second Fc polypeptides, respectively. In some embodiments wherein the first and second Fc polypeptides meet at an interface, the interface of the first Fc polypeptide (sequence) comprises a protuberance which is positionable in a cavity in the interface of the second Fc polypeptide (sequence). In one embodiment, the second Fc polypeptide has been altered from a template/original polypeptide to encode the cavity or the first Fc polypeptide has been altered from a template/original polypeptide to encode the protuberance, or both. In one embodiment, the second Fc polypeptide has been altered from a template/original polypeptide to encode the cavity and the first Fc polypeptide has been altered from a template/original polypeptide to encode the protuberance, or both. In one embodiment, the interface of the first Fc polypeptide comprises a protuberance which is positionable in a cavity in the interface of the second Fc polypeptide, wherein the protuberance or cavity, or both, have been introduced into the interface of the first and second Fc polypeptides, respectively.

In one embodiment, the protuberance and cavity each comprises a naturally occurring amino acid residue. In one embodiment, the Fc polypeptide comprising the protuberance is generated by replacing an original residue from the interface of a template/original polypeptide with an import residue having a larger side chain volume than the original residue. In one embodiment, the Fc polypeptide comprising the protuberance is generated by a method comprising a step wherein nucleic acid encoding an original residue from the interface of said polypeptide is replaced with nucleic acid encoding an import residue having a larger side chain volume than the original. In one embodiment, the original residue is threonine. In one embodiment, the import residue is arginine (R). In one embodiment, the import residue is phenylalanine (F). In one embodiment, the import residue is tyrosine (Y). In one embodiment, the import residue is tryptophan (W). In one embodiment, the import residue is R, F, Y or W. In one embodiment, a protuberance is generated by replacing two or more residues in a template/original polypeptide. In one embodiment, the Fc polypeptide comprising a protuberance comprises replacement of threonine at position 366 with tryptophan, amino acid numbering according to the EU numbering scheme of Kabat et al. (pp. 688-696 in Sequences of proteins of immunological interest, 5th ed., Vol. 1 (1991; NIH, Bethesda, Md.)).

In some embodiments, the Fc polypeptide comprising a cavity is generated by replacing an original residue in the interface of a template/original polypeptide with an import residue having a smaller side chain volume than the original residue. For example, the Fc polypeptide comprising the cavity may be generated by a method comprising a step wherein nucleic acid encoding an original residue from the interface of said polypeptide is replaced with nucleic acid encoding an import residue having a smaller side chain volume than the original. In one embodiment, the original residue is threonine. In one embodiment, the original residue is leucine. In one embodiment, the original residue is tyrosine. In one embodiment, the import residue is not cysteine (C). In one embodiment, the import residue is alanine (A). In one embodiment, the import residue is serine (S). In one embodiment, the import residue is threonine (T). In one embodiment, the import residue is valine (V). A cavity can be generated by replacing one or more original residues of a template/original polypeptide. For example, in one embodiment, the Fc polypeptide comprising a cavity comprises replacement of two or more original amino acids selected from the group consisting of threonine, leucine and tyrosine. In one embodiment, the Fc polypeptide comprising a cavity comprises two or more import residues selected from the group consisting of alanine, serine, threonine and valine. In some embodiments, the Fc polypeptide comprising a cavity comprises replacement of two or more original amino acids selected from the group consisting of threonine, leucine and tyrosine, and wherein said original amino acids are replaced with import residues selected from the group consisting of alanine, serine, threonine and valine. In one embodiment, the Fc polypeptide comprising a cavity comprises replacement of threonine at position 366 with serine, amino acid numbering according to the EU numbering scheme of Kabat et al. supra. In one embodiment, the Fc polypeptide comprising a cavity comprises replacement of leucine at position 368 with alanine, amino acid numbering according to the EU numbering scheme of Kabat et al. supra. In one embodiment, the Fc polypeptide comprising a cavity comprises replacement of tyrosine at position 407 with valine, amino acid numbering according to the EU numbering scheme of Kabat et al. supra. In one embodiment, the Fc polypeptide comprising a cavity comprises two or more amino acid replacements selected from the group consisting of T366S, L368A and Y407V, amino acid numbering according to the EU numbering scheme of Kabat et al. supra. In some embodiments of these antibody fragments, the Fc polypeptide comprising the protuberance comprises replacement of threonine at position 366 with tryptophan, amino acid numbering according to the EU numbering scheme of Kabat et al. supra.

The Fc sequence of the first and second heavy chain polypeptides may or may not be identical, provided they are capable of dimerizing to form an Fc region (as defined herein). A first Fc polypeptide is generally contiguously linked to one or more domains of an immunoglobulin heavy chain in a single polypeptide, for example with hinge, constant and/or variable domain sequences. In one embodiment, the first Fc polypeptide comprises at least a portion (including all) of a hinge sequence, at least a portion (including all) of a CH2 domain and/or at least a portion (including all) of a CH3 domain. In one embodiment, the first Fc polypeptide comprises the hinge sequence and the CH2 and CH3 domains of an immunoglobulin. In one embodiment, the second Fc polypeptide comprises at least a portion (including all) of a hinge sequence, at least a portion (including all) of a CH2 domain and/or at least a portion (including all) of a CH3 domain. In one embodiment, the second Fc polypeptide comprises the hinge sequence and the CH2 and CH3 domains of an immunoglobulin. In one embodiment, an antibody of the invention comprises first and second Fc polypeptides each of which comprising at least a portion of at least one antibody constant domain. In one embodiment, the antibody constant domain is a CH2 and/or CH3 domain. In any of the embodiments of an antibody of the invention that comprises a constant domain, the antibody constant domain can be from any immunoglobulin class, for example an IgG. The immunoglobulin source can be of any suitable species of origin (e.g., an IgG may be human IgG₁) or of synthetic form.

In one embodiment, a first light chain polypeptide and a second light chain polypeptide in a first and second target molecule binding arm, respectively, of an antibody of the invention comprise different/distinct antigen binding determinants (e.g., different/distinct variable domain sequences). In one embodiment, a first light chain polypeptide and a second light chain polypeptide in a first and second target molecule binding arm, respectively, of an antibody of the invention comprise the same (i.e., a common) antigen binding determinant e.g., the same variable domain sequence).

In one embodiment, an antibody of the invention comprises both (a) a variant hinge region (as described herein), and (b) a heavy chain interface that enhances heterodimerization (as described herein).

First and second host cells in methods of the invention can be cultured in any setting that permits expression and isolation of the polypeptides of interest. For example, in one embodiment, the first host cell and the second host cell in a method of the invention are grown as separate cell cultures. In another embodiment, the first host cell and the second host cell in a method of the invention are grown as a mixed culture comprising both host cells.

In some instances, it may be beneficial to control expression levels of polypeptides in methods of the invention. Various methods are known in the art for achieving the appropriate level of control. For example, in one embodiment of methods of the invention, nucleic acids encoding the polypeptides are operably linked to translational initiation regions (TIRs) of appropriate strength to control expression levels. In one embodiment, the TIRs are of approximately equal relative strength. For example, in one embodiment, the TIRs for expression of the polypeptides in a first host cell and a second host cell have a relative strength of about 1:1. In another embodiment, the TIRs for expression of the polypeptides in a first host cell and a second host cell have a relative strength of about 2:2.

It is to be understood that methods of the invention can include other steps which generally are routine steps evident for initiating and/or completing the process encompassed by methods of the invention as described herein. For example, in one embodiment, step (a) of a method of the invention is preceded by a step wherein nucleic acid encoding first heavy and light chain polypeptides is introduced into a first host cell, and nucleic acid encoding second heavy and light chain polypeptides is introduced into a second host cell. In one embodiment, methods of the invention further comprise a step of purifying heteromultimeric molecules having binding specificity to at least two distinct target molecules. In one embodiment, no more than about 10, 15, or 20% of isolated polypeptides are present as monomers or heavy-light chain dimers prior to the step of purifying the heteromultimers.

Polypeptides in methods of the invention can be incubated at a variety of temperature. For example, in one embodiment, polypeptide annealing step (e.g., step (d) in some methods of the invention) in a method of the invention comprises incubating mixture of isolated polypeptides at room temperature. In another embodiment, polypeptide annealing step (e.g., step (d) in some methods of the invention) in a method of the invention comprises heating mixture of isolated polypeptides, e.g. to at least about 40° C., to at least about 50° C. In one embodiment, the mixture is heated to between about 40° C. and 60° C. In one embodiment, the mixture is heated to between about 40° C. and 65° C. In one embodiment, the mixture is heated to between about 37° C. and 65° C. In one embodiment, the mixture is at about 50° C. In one embodiment, polypeptide annealing step (e.g., step (d) in some methods of the invention) in a method of the invention comprises heating the mixture of isolated polypeptides for at least about 2 minute, 4 min, 6 min, 8 min, 10 min, 15 min, 30 min, 45 min, 60 min, 75 min, 120 min. In one embodiment, polypeptide annealing step (e.g., step (d) in some methods of the invention) in a method of the invention comprises heating the mixture of isolated polypeptides for between 2 and 75, 5 and 120 min, 6 and 60, 8 and 45, 10 and 30, or 13 and 30 min. In one embodiment, polypeptide annealing step (e.g., step (d) in some methods of the invention) in a method of the invention comprises heating the mixture of isolated polypeptides for about 5 min, for about 10 min, for about 15 min., for about 20 min., for about 25 min., for about 30 min., for about 60 min., for about 75 min., or for about 120 min. In one embodiment of a method of the invention, the mixture of polypeptides is cooled, e.g. to 4° C., after heating.

In some instances, polypeptide annealing step of methods of the invention are carried out under pH-buffered conditions. For example, in one embodiment, in vitro polypeptide annealing step in a method of the invention (e.g., step (d) of some methods of the invention) comprises incubating the mixture of isolated polypeptides at a pH at or between about 4 to about 11. In one embodiment, the pH is about 5.5. In one embodiment, the pH is about 7.5.

In some instances, polypeptide annealing step of methods of the invention comprises incubating the mixture of isolated polypeptides in a denaturant, such as urea.

In many instances, chemical conjugation steps as used in some art methods are undesirable and/or create undesirable properties. Therefore, in some embodiments, methods of the invention do not include chemical conjugation between a first and second heavy chain polypeptide.

Methods of the invention are capable of generating heteromultimeric molecules at high homogeneity. According, the invention provides methods wherein at least about 60, 70, 75, 80, 85, 90, 95, 96, 97, 98, 99% of polypeptides are in a complex comprising a first heavy and light chain polypeptide pair and a second heavy and light chain polypeptide pair. In one embodiment, the invention provides methods wherein between about 60 and 99%, 70 and 98%, 75 and 97%, 80 and 96%, 85 and 96%, or 90 and 95% of polypeptides are in a complex comprising a first heavy and light chain polypeptide pair and a second heavy and light chain polypeptide pair.

In some embodiments of methods of the invention comprising first and second heavy-light chain polypeptide pairs, the first and second heavy-light chain pairs each comprises heavy and light chains covalently linked (e.g., disulfide linked) to each other. In some instances, the amount of first and second polypeptide pairs are provided at specific ratios, e.g. in approximately equimolar amount (ratio) in the polypeptide annealing/combining step. In other embodiments, the ratio of the first pair to second pair is about 1.2:1; 1.3:1; 1.4:1; or 1.5:1 in the annealing/combining step. In other embodiments, the ratio of the second pair to first pair is about 1.2:1; 1.3:1; 1.4:1; or 1.5:1 in the annealing/combining step.

To facilitate purification of a desired heteromultimer in some methods of the invention, it may be desirable to keep the pI value differential between a first polypeptide pair and a second polypeptide pair at at least 0.5. As would be evident to one skilled in the art, polypeptide pI values can be changed by routine techniques, such as selective substitutions in, for example, a CDR or FR sequence without substantially affecting antigen binding and/or immunogenicity.

In one embodiment, an antibody of the invention is selected from the group consisting of IgG, IgE, IgA, IgM and IgD. In some embodiments, the hinge region of an antibody of the invention is preferably of an immunoglobulin selected from the group consisting of IgG, IgA and IgD. For example, in some embodiments, an antibody or hinge region of an antibody is of IgG, which in some embodiments is IgG1 or IgG2 (e.g., IgG2a or IgG2b). In some embodiments, an antibody of the invention is selected from the group consisting of IgG, IgA and IgD. In one embodiment, the antibody is of human, humanized, chimeric or non-human (e.g., murine) origin.

Antibodies of the invention find a variety of uses in a variety of settings. In one example, an antibody of the invention is a therapeutic antibody. In another example, an antibody of the invention is an agonist antibody. In another example, an antibody of the invention is an antagonistic antibody. An antibody of the invention may also be a diagnostic antibody. In yet another example, an antibody of the invention is a blocking antibody. In another example, an antibody of the invention is a neutralizing antibody.

In one aspect, the invention provides methods of treating or delaying a disease in a subject, said methods comprising administering an antibody of the invention to said subject. In one embodiment, the disease is cancer. In another embodiment, the disease is associated with dysregulation of angiogenesis. In another embodiment, the disease is an immune disorder, such as rheumatoid arthritis, immune thrombocytopenic purpura, systemic lupus erythematosus, etc.

Antibodies of the invention generally are capable of binding, preferably specifically, to antigens. Such antigens include, for example, tumor antigens, cell survival regulatory factors, cell proliferation regulatory factors, molecules associated with (e.g., known or suspected to contribute functionally to) tissue development or differentiation, cell surface molecules, lymphokines, cytokines, molecules involved in cell cycle regulation, molecules involved in vasculogenesis and molecules associated with (e.g., known or suspected to contribute functionally to) angiogenesis. An antigen to which an antibody of the invention is capable of binding may be a member of a subset of one of the above-mentioned categories, wherein the other subset(s) of said category comprise other molecules/antigens that have a distinct characteristic (with respect to the antigen of interest). An antigen of interest may also be deemed to belong to two or more categories. In one embodiment, the invention provides an antibody that binds, preferably specifically, a tumor antigen that is not a cell surface molecule. In one embodiment, a tumor antigen is a cell surface molecule, such as a receptor polypeptide. In another example, in some embodiments, an antibody of the invention binds, preferably specifically, a tumor antigen that is not a cluster differentiation factor. In another example, an antibody of the invention is capable of binding, preferably specifically, to a cluster differentiation factor, which in some embodiments is not, for example, CD3 or CD4. In some embodiments, an antibody of the invention is an anti-VEGF antibody.

Antibodies may be modified to enhance and/or add additional desired characteristics. Such characteristics include biological functions such as immune effector functions, a desirable in vivo half life/clearance, bioavailability, biodistribution or other pharmacokinetic characteristics. Such modifications are well known in the art and can also be determined empirically, and may include modifications by moieties that may or may not be peptide-based. For example, antibodies may be glycosylated or aglycosylated, generally depending at least in part on the nature of the host cell. Preferably, antibodies of the invention are aglycosylated. An aglycosylated antibody produced by a method of the invention can subsequently be glycosylated by, for example, using in vitro glycosylation methods well known in the art. As described above and herein, antibodies of the invention can be produced in a prokaryotic cell, such as, for example, E. coli. E. coli-produced antibodies are generally aglycosylated and lack the biological functions normally associated with glycosylation profiles found in mammalian host cell (e.g., CHO) produced antibodies.

The invention also provides immunoconjugates comprising an antibody of the invention conjugated with a heterologous moiety. Any heterologous moiety would be suitable so long as its conjugation to the antibody does not substantially reduce a desired function and/or characteristic of the antibody. For example, in some embodiments, an immunoconjugate comprises a heterologous moiety which is a cytotoxic agent. In some embodiments, said cytotoxic agent is selected from the group consisting of a radioactive isotope, a chemotherapeutic agent and a toxin. In some embodiments, said toxin is selected from the group consisting of calichemicin, maytansine and trichothene. In some embodiments, an immunoconjugate comprises a heterologous moiety which is a detectable marker. In some embodiments, said detectable marker is selected from the group consisting of a radioactive isotope, a member of a ligand-receptor pair, a member of an enzyme-substrate pair and a member of a fluorescence resonance energy transfer pair.

In one aspect, the invention provides compositions comprising an antibody of the invention and a carrier, which in some embodiments is pharmaceutically acceptable.

In another aspect, the invention provides compositions comprising an immunoconjugate as described herein and a carrier, which in some embodiments is pharmaceutically acceptable.

In one aspect, the invention provides a composition comprising a population of multispecific antibodies of the invention. As would be evident to one skilled in the art, generally such a composition would not be completely (i.e., 100%) homogeneous. However, as described herein, methods of the invention are capable of producing a substantially homogeneous population of multispecific antibodies. For example, the invention provides a composition comprising antibodies, wherein at least 80, 85, 90, 95, 96, 97, 98, 99% of said antibodies are a multispecific antibody of the invention as described herein.

In one aspect, the invention provides a composition comprising a reaction mixture comprising a disulfide linked first pair of heavy and light chain polypeptides and a disulfide linked second pair of heavy and light chain polypeptides, wherein at least 50%, 55%, 60%, 65%, 70% of the first pair and second pair are multimerized (e.g., heterodimerized) to form a multispecific (e.g., bispecific) antibody.

In one aspect, the invention provides a cell culture comprising a mix of a first host cell and a second host cell, wherein the first host cell comprises nucleic acid encoding a first pair of heavy and light chain polypeptides, and the second host cell comprises nucleic acid encoding a second pair of heavy and light chain polypeptides, and wherein the two pairs have different target binding specificities. In one aspect, the invention provides a cell culture comprising a mix of a first host cell and a second host cell, wherein the first host cell expresses a first pair of heavy and light chain polypeptides, and the second host cell expresses a second pair of heavy and light chain polypeptides, and wherein the two pairs have different target binding specificities.

In another aspect, the invention provides articles of manufacture comprising a container and a composition contained therein, wherein the composition comprises a molecule (e.g. an antibody) of the invention. In another aspect, the invention provides articles of manufacture comprising a container and a composition contained therein, wherein the composition comprises an immunoconjugate as described herein. In some embodiments, these articles of manufacture further comprise instruction for using said composition.

In yet another aspect, the invention provides polynucleotides encoding an antibody of the invention. In still another aspect, the invention provides polynucleotides encoding an immunoconjugate as described herein.

In one aspect, the invention provides recombinant vectors for expressing a molecule (e.g., an antibody) of the invention. In another aspect, the invention provides recombinant vectors for expressing an immunoconjugate of the invention.

In one aspect, the invention provides host cells comprising a polynucleotide or recombinant vector of the invention. In one embodiment, a host cell is a mammalian cell, for example a Chinse Hamster Ovary (CHO) cell. In one embodiment, a host cell is a prokaryotic cell. In some embodiments, a host cell is a gram-negative bacterial cell, which in some embodiments is E. coli. Host cells of the invention may further comprise a polynucleotide or recombinant vector encoding a molecule the expression of which in a host cell enhances yield of an antibody in a method of the invention. For example, such molecule can be a chaperone protein. In one embodiment, said molecule is a prokaryotic polypeptide selected from the group consisting of DsbA, DsbC, DsbG and FkpA. In some embodiments, said polynucleotide or recombinant vector encodes both DsbA and DsbC. In some embodiments, an E. coli host cell is of a strain deficient in endogenous protease activities. In some embodiments, the genotype of an E. coli host cell is that of an E. coli strain that lacks degP and prc genes and harbors a mutant spr gene.

In one aspect, the invention provides use of a molecule (e.g., an antibody) of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a nucleic acid of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of an expression vector of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a host cell of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of an article of manufacture of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a kit of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

### BRIEF DESCRIPTION OF DRAWINGS

FIG. 1 depicts anti-Fab Western blot results for p5A6.11.Knob (knob anti-Fcγ-RIIb) and p22E7.11.Hole (hole anti-IgE-R) antibody component expression.

FIG. 2 depicts anti-Fc Western blot results for p5A6.11.Knob (knob anti-Fcγ-RIIb) and p22E7.11.Hole (hole anti-IgE-R) antibody component expression.

FIG. 3 depicts anti-Fab Western blot results for expression of antibody components with wild type or variant hinge sequences.

FIG. 4 depicts anti-Fc Western blot results for expression of antibody components with wild type or variant hinge sequences.

FIG. 5 depicts isoelectric focusing analysis of 5A6Knob, 22E7Hole, mixed 5A6Knob and 22E7Hole (all heavy chains having variant hinge as described) at room temperature, and the mixture heated to 50° C. for 5 minutes.

FIG. 6 depicts FcγRIIb affinity column flow-throughs for 5A6Knob/22E7Hole bispecific, 22E7Hole, and 5A6Knob antibodies (all heavy chains having variant hinge as described).

FIG. 7 isoelectric focusing analysis of 5A6Knob, 22E7Hole, and 5A6Knob and 22E7Hole mixture heated to 50° C. for 10 minutes (all heavy chains having variant hinge as described).

FIG. 8 depicts a nucleic acid sequence encoding the alkaline phosphatase promoter (phoA), STII signal sequence and the entire (variable and constant domains) light chain of the 5A6 antibody.

FIG. 9 depicts a nucleic acid sequence encoding the last 3 amino acids of the STII signal sequence and approximately 119 amino acids of the murine heavy variable domain of the 5A6 antibody.

FIG. 10 depicts a nucleic acid sequence encoding the alkaline phosphatase promoter (phoA), STII signal sequence and the entire (variable and constant domains) light chain of the 22E7 antibody.

FIG. 11 depicts a nucleic acid sequence encoding the last 3 amino acids of the STII signal sequence and approximately 123 amino acids of the murine heavy variable domain of the 22E7 antibody.

### MODES FOR CARRYING OUT THE INVENTION

The invention provides improved methods, compositions, kits and articles of manufacture for generating heteromultimeric complex molecules such as antibodies. The invention enables generation of heteromultimeric at pragmatic yields and desirable purity. The invention makes possible the efficient and commercially viable production of complex molecules that in turn can be used for treating pathological conditions in which use of a molecule that is multispecific in nature and highly stable is highly desirable and/or required. Details of methods, compositions, kits and articles of manufacture of the invention are provided herein.

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988).

Definitions

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR.sub.2 (“amidate”), P(O)R, P(O)OR′, CO or CH.sub.2 (“formacetal”), in which each R or R′ is independently H or substituted or unsubstituted alkyl (1-20 C.) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The terms “antibody” and “immunoglobulin” are used interchangeably in the broadest sense and include monoclonal antibodies (e.g., full length or intact monoclonal antibodies), polyclonal antibodies, multivalent antibodies, multispecific antibodies (e.g., bispecific antibodies so long as they exhibit the desired biological activity) and antibody fragments as described herein. An antibody can be human, humanized and/or affinity matured.

“Antibody fragments” comprise only a portion of an intact antibody, wherein the portion preferably retains at least one, preferably most or all, of the functions normally associated with that portion when present in an intact antibody.

The phrase “antigen binding arm”, “target molecule binding arm”, and variations thereof, as used herein, refers to a component part of an antibody of the invention that has an ability to specifically bind a target molecule of interest. Generally and preferably, the antigen binding arm is a complex of immunoglobulin polypeptide sequences, e.g., CDR and/or variable domain sequences of an immunoglobulin light and heavy chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As used herein, the term “immunoadhesin” designates antibody-like molecules which combine the “binding domain” of a heterologous protein (an “adhesin”, e.g. a receptor, ligand or enzyme) with the effector component of immunoglobulin constant domains. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The immunoglobulin constant domain sequence in the immunoadhesin may be obtained from any immunoglobulin, such as IgG₁, IgG₂, IgG₃, or IgG₄ subtypes, IgA, IgE, IgD or IgM.

A “heteromultimer”, “heteromultimeric complex”, or “heteromultimeric polypeptide” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where polypeptides in addition to the first and second polypeptide are present.

As used herein, “polypeptide” refers generally to peptides and proteins having more than about ten amino acids.

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region. An Fc polypeptide may be obtained from any suitable immunoglobulin, such as IgG₁, IgG₂, IgG₃, or IgG₄ subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The “hinge region,” “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

The term “cistron,” as used herein, is intended to refer to a genetic element broadly equivalent to a translational unit comprising the nucleotide sequence coding for a polypeptide chain and adjacent control regions. “Adjacent control regions” include, for example, a translational initiation region (TIR; as defined herein below) and a termination region.

The “translation initiation region” or TIR, as used herein refers to a nucleic acid region providing the efficiency of translational initiation of a gene of interest. In general, a TIR within a particular cistron encompasses the ribosome binding site (RBS) and sequences 5′ and 3′ to RBS. The RBS is defined to contain, minimally, the Shine-Dalgarno region and the start codon (AUG). Accordingly, a TIR also includes at least a portion of the nucleic acid sequence to be translated. In some embodiments, a TIR of the invention includes a secretion signal sequence encoding a signal peptide that precedes the sequence coding for the light or heavy chain within a cistron. A TIR variant contains sequence variants (particularly substitutions) within the TIR region that alter the property of the TIR, such as its translational strength as defined herein below. Preferably, a TIR variant of the invention contains sequence substitutions within the first 2 to about 14, preferably about 4 to 12, more preferably about 6 codons of the secretion signal sequence that precedes the sequence coding for the light or heavy chain within a cistron.

The term “translational strength” as used herein refers to a measurement of a secreted polypeptide in a control system wherein one or more variants of a TIR is used to direct secretion of a polypeptide and the results compared to the wild-type TIR or some other control under the same culture and assay conditions. Without being limited to any one theory, “translational strength” as used herein can include, for example, a measure of mRNA stability, efficiency of ribosome binding to the ribosome binding site, and mode of translocation across a membrane.

“Secretion signal sequence” or “signal sequence” refers to a nucleic acid sequence coding for a short signal peptide that can be used to direct a newly synthesized protein of interest through a cellular membrane, for example the inner membrane or both inner and outer membranes of prokaryotes. As such, the protein of interest such as the immunoglobulin light or heavy chain polypeptide may be secreted into the periplasm of prokaryotic host cells or into the culture medium. The signal peptide encoded by the secretion signal sequence may be endogenous to the host cells, or they may be exogenous, including signal peptides native to the polypeptide to be expressed. Secretion signal sequences are typically present at the amino terminus of a polypeptide to be expressed, and are typically removed enzymatically between biosynthesis and secretion of the polypeptide from the cytoplasm. Thus, the signal peptide is usually not present in a mature protein product.

A “blocking” antibody or an “antagonist” antibody is one which inhibits or reduces biological activity of the antigen it binds.

An “agonist antibody”, as used herein, is an antibody which mimics at least one of the functional activities of a polypeptide of interest.

A “tumor antigen,” as used herein, includes the meaning known in the art, which includes any molecule that is differentially expressed on a tumor cell compared to a normal cell. In some embodiments, the molecule is expressed at a detectably or significantly higher or lower level in a tumor cell compared to a normal cell. In some embodiments, the molecule exhibits a detectably or significantly higher or lower level of biological activity in a tumor cell compared to a normal cell. In some embodiments, the molecule is known or thought to contribute to a tumorigenic characteristic of the tumor cell. Numerous tumor antigens are known in the art. Whether a molecule is a tumor antigen can also be determined according to techniques and assays well known to those skilled in the art, such as for example clonogenic assays, transformation assays, in vitro or in vivo tumor formation assays, gel migration assays, gene knockout analysis, etc.

A “disorder” is any condition that would benefit from treatment with an antibody or method of the invention. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic and other angiogenesis-related disorders.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma (e.g., non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

An “autoimmune disease” herein is a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

Dysregulation of angiogenesis can lead to many disorders that can be treated by compositions and methods of the invention. These disorders include both non-neoplastic and neoplastic conditions. Neoplastics include but are not limited those described above. Non-neoplastic disorders include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result. A “therapeutically effective amount” of an antibody of the invention may vary according to factors such as the disease state, age, sex, and weight of the individual, and the ability of the antibody to elicit a desired response in the individual. A therapeutically effective amount is also one in which any toxic or detrimental effects of the antibody are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The phrase “substantially similar”, “substantially identical”, “substantially the same”, and variations thereof, as used herein, denotes a sufficiently high degree of similarity between two numeric values (generally one associated with an antibody of the invention and the other associated with its reference counterpart) such that one of skill in the art would consider the difference between the two values to be of little or no biological significance within the context of the biological, physical or quantitation characteristic measured by said values. The difference between said two values is preferably less than about 50%, preferably less than about 40%, preferably less than about 30%, preferably less than about 20%, preferably less than about 10% as a function of the value for the reference counterpart.

“Complement dependent cytotoxicity” and “CDC” refer to the lysing of a target in the presence of complement. The complement activation pathway is initiated by the binding of the first component of the complement system (Clq) to a molecule (e.g. an antibody) complexed with a cognate antigen.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma II and calicheamicin omegaI1 (see, e.g., Agnew, Chem. Intl. Ed. Engl., 33:183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA®) pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase I inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth of a cell whose growth is dependent upon activation of a molecule targeted by a molecule of the invention either in vitro or in vivo. Thus, the growth inhibitory agent may be one which significantly reduces the percentage of target molecule-dependent cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL(®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

Except where indicated otherwise by context, the terms “first” polypeptide and “second” polypeptide, and variations thereof, are merely generic identifiers, and are not to be taken as identifying a specific or a particular polypeptide or component of antibodies of the invention.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimer, and thereby favor heteromultimer formation over homomultimer formation, for example. The protuberance may exist in the original interface or may be introduced synthetically (e.g. by altering nucleic acid encoding the interface). Normally, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The upper limit for the number of original residues which are replaced is the total number of residues in the interface of the first polypeptide. The side chain volumes of the various amino residues are shown in the following table.

TABLE 1
Properties of Amino Acid Residues

| | | | | Accessible |
|---|---|---|---|---|
| | | | | Surface |
| | One-Letter | MASS^{a} | VOLUME^{b} | Area^{c} |
| Amino Acid | Abbreviation | (daltons) | (Angstrom³) | (Angstrom²) |
| Alanine (Ala) | A | 71.08 | 88.6 | 115 |
| Arginine (Arg) | R | 156.20 | 173.4 | 225 |
| Asparagine (Asn) | N | 114.11 | 117.7 | 160 |
| Aspartic acid | D | 115.09 | 111.1 | 150 |
| (Asp) | | | | |
| Cysteine (Cys) | C | 103.14 | 108.5 | 135 |
| Glutamine (Gln) | Q | 128.14 | 143.9 | 180 |
| Glutamic acid | E | 129.12 | 138.4 | 190 |
| (Glu) | | | | |
| Glycine (Gly) | G | 57.06 | 60.1 | 75 |
| Histidine (His) | H | 137.15 | 153.2 | 195 |
| Isoleucine (Ile) | I | 113.17 | 166.7 | 175 |
| Leucine (Leu) | L | 113.17 | 166.7 | 170 |
| Lysine (Lys) | K | 128.18 | 168.6 | 200 |
| Methionine (Met) | M | 131.21 | 162.9 | 185 |
| Phenylalinine | F | 147.18 | 189.9 | 210 |
| (Phe) | | | | |
| Proline (Pro) | P | 97.12 | 122.7 | 145 |
| Serine (Ser) | S | 87.08 | 89.0 | 115 |
| Threonine (Thr) | T | 101.11 | 116.1 | 140 |
| Tryptophan (Trp) | W | 186.21 | 227.8 | 255 |
| Tyrosine (Tyr) | Y | 163.18 | 193.6 | 230 |
| Valine (Val) | V | 99.14 | 140.0 | 155 |

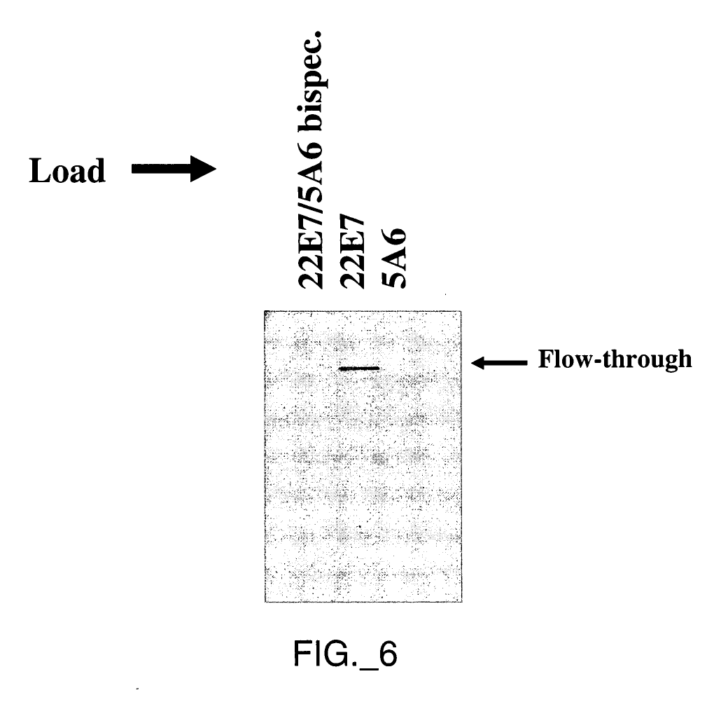

^{a}Molecular weight amino acid minus that of water. Values from Handbook of Chemistry and Physics, 43rd ed. Cleveland, Chemical Rubber Publishing Co., 1961.
^{b}Values from A. A. Zamyatnin, Prog. Biophys. Mol. Biol. 24: 107-123, 1972.
^{c}Values from C. Chothia, J. Mol. Biol. 105: 1-14, 1975. The accessible surface area is defined in FIGS. 6-20 of this reference.

The preferred import residues for the formation of a protuberance are generally naturally occurring amino acid residues and are preferably selected from arginine (R), phenylalanine (F), tyrosine (Y) and tryptophan (W). Most preferred are tryptophan and tyrosine. In one embodiment, the original residue for the formation of the protuberance has a small side chain volume, such as alanine, asparagine, aspartic acid, glycine, serine, threonine or valine.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). Normally, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. The upper limit for the number of original residues which are replaced is the total number of residues in the interface of the second polypeptide. The side chain volumes of the various amino residues are shown in Table 1 above. The preferred import residues for the formation of a cavity are usually naturally occurring amino acid residues and are preferably selected from alanine (A), serine (S), threonine (T) and valine (V). Most preferred are serine, alanine or threonine. In one embodiment, the original residue for the formation of the cavity has a large side chain volume, such as tyrosine, arginine, phenylalanine or tryptophan.

An “original” amino acid residue is one which is replaced by an “import” residue which can have a smaller or larger side chain volume than the original residue. The import amino acid residue can be a naturally occurring or non-naturally occurring amino acid residue, but preferably is the former. “Naturally occurring” amino acid residues are those residues encoded by the genetic code and listed in Table 1 above. By “non-naturally occurring” amino acid residue is meant a residue which is not encoded by the genetic code, but which is able to covalently bind adjacent amino acid residue(s) in the polypeptide chain. Examples of non-naturally occurring amino acid residues are norleucine, ornithine, norvaline, homoserine and other amino acid residue analogues such as those described in Ellman et al., Meth. Enzym. 202:301-336 (1991), for example. To generate such non-naturally occurring amino acid residues, the procedures of Noren et al. Science 244:182 (1989) and Ellman et al., supra can be used. Briefly, this involves chemically activating a suppressor tRNA with a non-naturally occurring amino acid residue followed by in vitro transcription and translation of the RNA. The method of the instant invention involves replacing at least one original amino acid residue, but more than one original residue can be replaced. Normally, no more than the total residues in the interface of the first or second polypeptide will comprise original amino acid residues which are replaced. Typically, original residues for replacement are “buried”. By “buried” is meant that the residue is essentially inaccessible to solvent. Generally, the import residue is not cysteine to prevent possible oxidation or mispairing of disulfide bonds.

The protuberance is “positionable” in the cavity which means that the spatial location of the protuberance and cavity on the interface of a first polypeptide and second polypeptide. respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the axis of the interface and have preferred conformations, the alignment of a protuberance with a corresponding cavity relies on modeling the protuberance/cavity pair based upon a three-dimensional structure such as that obtained by X-ray crystallography or nuclear magnetic resonance (NMR). This can be achieved using widely accepted techniques in the art.

By “original or template nucleic acid” is meant the nucleic acid encoding a polypeptide of interest which can be “altered” (i.e. genetically engineered or mutated) to encode a protuberance or cavity. The original or starting nucleic acid may be a naturally occurring nucleic acid or may comprise a nucleic acid which has been subjected to prior alteration (e.g. a humanized antibody fragment). By “altering” the nucleic acid is meant that the original nucleic acid is mutated by inserting, deleting or replacing at least one codon encoding an amino acid residue of interest. Normally, a codon encoding an original residue is replaced by a codon encoding an import residue. Techniques for genetically modifying a DNA in this manner have been reviewed in Mutagenesis: a Practical Approach, M. J. McPherson, Ed., (IRL Press, Oxford, UK. (1991), and include site-directed mutagenesis, cassette mutagenesis and polymerase chain reaction (PCR) mutagenesis, for example. By mutating an original/template nucleic acid, an original/template polypeptide encoded by the original/template nucleic acid is thus correspondingly altered.

The protuberance or cavity can be “introduced” into the interface of a first or second polypeptide by synthetic means, e.g. by recombinant techniques, in vitro peptide synthesis, those techniques for introducing non-naturally occurring amino acid residues previously described, by enzymatic or chemical coupling of peptides or some combination of these techniques. Accordingly, the protuberance or cavity which is “introduced” is “non-naturally occurring” or “non-native”, which means that it does not exist in nature or in the original polypeptide (e.g. a humanized monoclonal antibody).

Generally, the import amino acid residue for forming the protuberance has a relatively small number of “rotamers” (e.g. about 3-6). A “rotomer” is an energetically favorable conformation of an amino acid side chain. The number of rotomers of the various amino acid residues are reviewed in Ponders and Richards, J. Mol. Biol. 193:775-791 (1987).

“Isolated” heteromultimer means heteromultimer which has been identified and separated and/or recovered from a component of its natural cell culture environment. Contaminant components of its natural environment are materials which would interfere with diagnostic or therapeutic uses for the heteromultimer, and may include enzymes, hormones, and other proteinaceous or nonproteinaceous solutes. In some embodiments, the heteromultimer will be purified (1) to greater than 95% by weight of protein as determined by the Lowry method, or more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain.

The heteromultimers of the present invention are generally purified to substantial homogeneity. The phrases “substantially homogeneous”, “substantially homogeneous form” and “substantial homogeneity” are used to indicate that the product is substantially devoid of by-products originated from undesired polypeptide combinations (e.g. homomultimers). Expressed in terms of purity, substantial homogeneity means that the amount of by-products does not exceed 20%, 10%, or is below 5%, or is below 1%, or is below 0.5%, wherein the percentages are by weight.

The expression “control sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking can be accomplished by ligation at convenient restriction sites. If such sites do not exist, the synthetic oligonucleotide adaptors or linkers are used in accord with conventional practice.

Vectors, Host Cells and Recombinant Methods

For recombinant production of an antibody of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

Generating Antibodies Using Prokaryotic Host Cells:

Vector Construction

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM.TM.-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20:269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB strains) provide cytoplasm conditions that are favorable for disulfide bond formation, thereby permitting proper folding and assembly of expressed protein subunits. Proba and Pluckthun Gene, 159:203 (1995).

The present invention provides an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components.

One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5, 840,523. Based on the translational strength comparison, the desired individual TIRs are selected to be combined in the expression vector constructs of the invention.

Prokaryotic host cells suitable for expressing antibodies of the invention include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In one embodiment, gram-negative cells are used. In one embodiment, E. coli cells are used as hosts for the invention. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 Δfhu (ΔtonA) ptr3 lac lq lacL8 ΔompTΔ(nmpc-fepE) degP41] kan^{R} (U.S. Pat. No. 5,639,635). Other strains and derivatives thereof, such as E. coli 294 (ATCC 31,446), E. coli B, E. coli_{λ} 1776 (ATCC 31,537) and E. coli RV308(ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

Antibody Production

Host cells are transformed with the above-described expression vectors and cultured in conventional nutrient media modified as appropriate for inducing promoters, selecting transformants, or amplifying the genes encoding the desired sequences.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD₅₅₀ of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

Antibody Purification

In one embodiment, the antibody protein produced herein is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the full length antibody products of the invention. Protein A is a 41 kD cell wall protein from Staphylococcus aureas which binds with a high affinity to the Fc region of antibodies. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

Generating Antibodies Using Eukaryotic Host Cells:

The vector components generally include, but are not limited to, one or more of the following: a signal sequence, an origin of replication, one or more marker genes, an enhancer element, a promoter, and a transcription termination sequence.

(i) Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

(ii) Origin of Replication

Generally, an origin of replication component is not needed for mammalian expression vectors. For example, the SV40 origin may typically be used only because it contains the early promoter.

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

(iv) Promoter Component

Expression and cloning vectors usually contain a promoter that is recognized by the host organism and is operably linked to the antibody polypeptide nucleic acid. Promoter sequences are known for eukaryotes. Virtually alleukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of DNA encoding the antibody polypeptide of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody polypeptide-encoding sequence, but is preferably located at a site 5′ from the promoter.

(vi) Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem.102:255 (1980), U.S. Pat. Nos. 4,767,704; 4,657,866; 4,927,762; 4,560,655; or 5,122,469; WO 90/03430; WO 87/00195; or U.S. Patent Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Activity Assays

The antibodies of the present invention can be characterized for their physical/chemical properties and biological functions by various assays known in the art.

The purified immunoglobulins can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In certain embodiments of the invention, the immunoglobulins produced herein are analyzed for their biological activity. In some embodiments, the immunoglobulins of the present invention are tested for their antigen binding activity. The antigen binding assays that are known in the art and can be used herein include without limitation any direct or competitive binding assays using techniques such as western blots, radioimmunoassays, ELISA (enzyme linked immnosorbent assay), “sandwich” immunoassays, immunoprecipitation assays, fluorescent immunoassays, and protein A immunoassays. An illustrative antigen binding assay is provided below in the Examples section.

In one embodiment, the present invention contemplates an altered antibody that possesses some but not all effector functions, which make it a desired candidate for many applications in which the half life of the antibody in vivo is important yet certain effector functions (such as complement and ADCC) are unnecessary or deleterious. In certain embodiments, the Fc activities of the produced immunoglobulin are measured to ensure that only the desired properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. Nos. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). Clq binding assays may also be carried out to confirm that the antibody is unable to bind Clq and hence lacks CDC activity. To assess complement activation, a CDC assay, e.g. as described in Gazzano-Santoro et al., J. Immunol. Methods 202:163 (1996), may be performed. FcRn binding and in vivo clearance/half life determinations can also be performed using methods known in the art.

Humanized Antibodies

The present invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Antibody Variants

In one aspect, the invention provides antibody fragment comprising modifications in the interface of Fc polypeptides comprising the Fc region, wherein the modifications facilitate and/or promote heterodimerization. These modifications comprise introduction of a protuberance into a first Fc polypeptide and a cavity into a second Fc polypeptide, wherein the protuberance is positionable in the cavity so as to promote complexing of the first and second Fc polypeptides. Methods of generating antibodies with these modifications are known in the art, e.g., as described in U.S. Pat. No. 5,731,168.

In some embodiments, amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 2 under the heading of “preferred substitutions”. If such substitutions result in a change in biological activity, then more substantial changes, denominated “exemplary substitutions” in Table 2, or as further described below in reference to amino acid classes, may be introduced and the products screened.

TABLE 2

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

One type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of the immunoglobulin polypeptides of the invention, thereby generating a Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In accordance with this description and the teachings of the art, it is contemplated that in some embodiments, an antibody of the invention may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) Clq binding and/or Complement Dependent Cytotoxicity (CDC), e.g., as described in WO99/51642. See also Duncan & Winter Nature 322:73840 (1988); U.S. Pat. No. 5,648,260; U.S. Pat. No. 5,624,821; and WO94/29351 concerning other examples of Fc region variants.

Immunoconjugates

The invention also pertains to immunoconjugates comprising an antibody of the invention conjugated to a cytotoxic agent such as a chemotherapeutic agent (as defined and described herein above), toxin (e.g. a small molecule toxin or an enzymatically active toxin of bacterial, fungal, plant or animal origin, including fragments and/or variants thereof), or a radioactive isotope (i.e., a radioconjugate).

Conjugates of an antibody and one or more small molecule toxins, such as a calicheamicin, a maytansine (U.S. Pat. No. 5,208,020), a trichothene, and CC1065 are also contemplated herein.

In one embodiment of the invention, the antibody is conjugated to one or more maytansine molecules (e.g. about 1 to about 10 maytansine molecules per antibody molecule). Maytansine may, for example, be converted to May-SS-Me which may be reduced to May-SH3 and reacted with modified antibody (Chari et al. Cancer Research 52:127-131 (1992)) to generate a maytansinoid-antibody immunoconjugate.

Another immunoconjugate of interest comprises an immunoglobulin conjugated to one or more calicheamicin molecules. The calicheamicin family of antibiotics are capable of producing double-stranded DNA breaks at sub-picomolar concentrations. Structural analogues of calicheamicin which may be used include, but are not limited to, y₁^{I}, α₂^{I}, α₃^{I}, N-acetyl-γ₁^{I}, PSAG and θ^{I}₁, (Hinman et al. Cancer Research 53:3336-3342 (1993) and Lode et al. Cancer Research 58:2925-2928 (1998)). See, also, U.S. Pat. Nos. 5,714,586; 5,712,374; 5,264,586; and 5,773,001.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an immunoglobulin of the invention and a compound with nucleolytic activity (e.g. a ribonuclease or a DNA endonuclease such as a deoxyribonuclease; DNase).

A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

Conjugates of the immunoglobulin of the invention and cytotoxic agent may be made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52:127-131 (1992)) may be used.

Alternatively, a fusion protein comprising the immunoglobulin and cytotoxic agent may be made, e.g. by recombinant techniques or peptide synthesis.

In yet another embodiment, an immunoglobulin of the invention may be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

Antibody Derivatives

The antibodies of the present invention can be further modified to contain additional nonproteinaceous moieties that are known in the art and readily available. Preferably, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Antigen Specificity

The present invention is applicable to antibodies of any appropriate antigen binding specificity. Preferably, the antibodies used in methods of the invention are specific to antigens that are biologically important polypeptides. More preferably, the antibodies of the invention are useful for therapy or diagnosis of diseases or disorders in a mammal. Antibodies of the invention include, but are not limited to blocking antibodies, agonist antibodies, neutralizing antibodies or antibody conjugates. Non-limiting examples of therapeutic antibodies include anti-c-met, anti-VEGF, anti-IgE, anti-CD11, anti-CD18, anti-CD40, anti-tissue factor (TF), anti-HER2, and anti-TrkC antibodies. Antibodies directed against non-polypeptide antigens (such as tumor-associated glycolipid antigens) are also contemplated.

Where the antigen is a polypeptide, it may be a transmembrane molecule (e.g. receptor) or a ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD3, CD4, CD8, CD19, CD20 and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins such as CD11a, CD11b, CD11c, CD18, an ICAM, VLA4 and VCAM; a tumor associated antigen such as HER2, HER3 or HER4 receptor; and fragments of any of the above-listed polypeptides.

Antigens for antibodies encompassed by one embodiment of the present invention include CD proteins such as CD3, CD4, CD8, CD19, CD20, CD34, and CD46; members of the ErbB receptor family such as the EGF receptor, HER2, HER3 or HER4 receptor; cell adhesion molecules such as LFA-1, Mac1, p150.95, VLA4, ICAM-1, VCAM, α4/β7 integrin, and αv/β3 integrin including either α or β subunits thereof (e.g. anti-CD11a, anti-CD18 or anti-CD11b antibodies); growth factors such as VEGF; tissue factor (TF); TGF-β; alpha interferon (α-IFN); an interleukin, such as IL-8; IgE; blood group antigens Apo2, death receptor; flk2/flt3 receptor; obesity (OB) receptor; mpl receptor; CTLA-4; protein C etc. In some embodiments, targets herein are VEGF, TF, CD19, CD20, CD40, TGF-β, CD11a, CD18, Apo2 and C24.

In some embodiments, an antibody of the invention is capable of binding specifically to a tumor antigen. In some embodiments, an antibody of the invention is capable of binding specifically to a tumor antigen wherein the tumor antigen is not a cluster differentiation factor (i.e., a CD protein). In some embodiments, an antibody of the invention is capable of binding specifically to a CD protein. In some embodiments, an antibody of the invention is capable of binding specifically to a CD protein other than CD3 or CD4. In some embodiments, an antibody of the invention is capable of binding specifically to a CD protein other than CD19 or CD20. In some embodiments, an antibody of the invention is capable of binding specifically to a CD protein other than CD40. In some embodiments, an antibody of the invention is capable of binding specifically to CD19 or CD20. In some embodiments, an antibody of the invention is capable of binding specifically to CD40. In some embodiments, an antibody of the invention is capable of binding specifically to CD11. In one embodiment, an antibody of the invention binds an antigen that is not expressed in an immune cell. In one embodiment, an antibody of the invention binds an antigen that is not expressed in T cells. In one embodiment, an antibody of the invention binds an antigen that is not expressed in B cells.

In one embodiment, an antibody of the invention is capable of binding specifically to a cell survival regulatory factor. In some embodiments, an antibody of the invention is capable of binding specifically to a cell proliferation regulatory factor. In some embodiments, an antibody of the invention is capable of binding specifically to a molecule involved in cell cycle regulation. In other embodiments, an antibody of the invention is capable of binding specifically to a molecule involved in tissue development or cell differentiation. In some embodiments, an antibody of the invention is capable of binding specifically to a cell surface molecule. In some embodiments, an antibody of the invention is capable of binding to a tumor antigen that is not a cell surface receptor polypeptide.

In one embodiment, an antibody of the invention is capable of binding specifically to a lymphokine. In another embodiment, an antibody of the invention is capable of binding specifically to a cytokine.

In one embodiment, antibodies of the invention are capable of binding specifically to a molecule involved in vasculogenesis. In another embodiment, antibodies of the invention are capable of binding specifically to a molecule involved in angiogenesis.

Soluble antigens or fragments thereof, optionally conjugated to other molecules, can be used as immunogens for generating antibodies. For transmembrane molecules, such as receptors, fragments of these molecules (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

Pharmaceutical Formulations

Therapeutic formulations comprising an antibody of the invention are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Uses

Molecules of the invention may be used in, for example, in vitro, ex vivo and in vivo therapeutic methods. The invention provides various methods based on using one or more of these molecules. In certain pathological conditions, it is necessary and/or desirable to utilize multispecific antibodies. The invention provides these antibodies, which can be used for a variety of purposes, for example as therapeutics, prophylactics and diagnostics. For example, the invention provides methods of treating a disease, said methods comprising administering to a subject in need of treatment an antibody of the invention, whereby the disease is treated. Any of the antibodies of the invention described herein can be used in therapeutic (or prophylactic or diagnostic) methods described herein.

Antibodies of the invention can be used as an antagonist to partially or fully block the specific antigen activity in vitro, ex vivo and/or in vivo. Moreover, at least some of the antibodies of the invention can neutralize antigen activity from other species. Accordingly, the antibodies of the invention can be used to inhibit a specific antigen activity, e.g., in a cell culture containing the antigen, in human subjects or in other mammalian subjects having the antigen with which an antibody of the invention cross-reacts (e.g. chimpanzee, baboon, marmoset, cynomolgus and rhesus, pig or mouse). In one embodiment, the antibody of the invention can be used for inhibiting antigen activities by contacting the antibody with the antigen such that antigen activity is inhibited. Preferably, the antigen is a human protein molecule.

In one embodiment, an antibody of the invention can be used in a method for inhibiting an antigen in a subject suffering from a disorder in which the antigen activity is detrimental, comprising administering to the subject an antibody of the invention such that the antigen activity in the subject is inhibited. Preferably, the antigen is a human protein molecule and the subject is a human subject. Alternatively, the subject can be a mammal expressing the antigen with which an antibody of the invention binds. Still further the subject can be a mammal into which the antigen has been introduced (e.g., by administration of the antigen or by expression of an antigen transgene). An antibody of the invention can be administered to a human subject for therapeutic purposes. Moreover, an antibody of the invention can be administered to a non-human mammal expressing an antigen with which the immunoglobulin cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration). Blocking antibodies of the invention that are therapeutically useful include, for example but not limited to, anti-c-met, anti-VEGF, anti-IgE, anti-CD11, anti-interferon and anti-tissue factor antibodies. The antibodies of the invention can be used to treat, inhibit, delay progression of, prevent/delay recurrence of, ameliorate, or prevent diseases, disorders or conditions associated with abnormal expression and/or activity of one or more antigen molecules, including but not limited to malignant and benign tumors; non-leukemias and lymphoid malignancies; neuronal, glial, astrocytal, hypothalamic and other glandular, macrophagal, epithelial, stromal and blastocoelic disorders; and inflammatory, angiogenic and immunologic disorders.

In one aspect, a blocking antibody of the invention is specific to a ligand antigen, and inhibits the antigen activity by blocking or interfering with the ligand-receptor interaction involving the ligand antigen, thereby inhibiting the corresponding signal pathway and other molecular or cellular events. The invention also features receptor-specific antibodies which do not necessarily prevent ligand binding but interfere with receptor activation, thereby inhibiting any responses that would normally be initiated by the ligand binding. The invention also encompasses antibodies that either preferably or exclusively bind to ligand-receptor complexes. An antibody of the invention can also act as an agonist of a particular antigen receptor, thereby potentiating, enhancing or activating either all or partial activities of the ligand-mediated receptor activation.

In certain embodiments, an immunoconjugate comprising an antibody conjugated with a cytotoxic agent is administered to the patient. In some embodiments, the immunoconjugate and/or antigen to which it is bound is/are internalized by the cell, resulting in increased therapeutic efficacy of the immunoconjugate in killing the target cell to which it binds. In one embodiment, the cytotoxic agent targets or interferes with nucleic acid in the target cell. Examples of such cytotoxic agents include any of the chemotherapeutic agents noted herein (such as a maytansinoid or a calicheamicin), a radioactive isotope, or a ribonuclease or a DNA endonuclease.

Antibodies of the invention can be used either alone or in combination with other compositions in a therapy. For instance, an antibody of the invention may be co-administered with another antibody, chemotherapeutic agent(s) (including cocktails of chemotherapeutic agents), other cytotoxic agent(s), anti-angiogenic agent(s), cytokines, and/or growth inhibitory agent(s). Where an antibody of the invention inhibits tumor growth, it may be particularly desirable to combine it with one or more other therapeutic agent(s) which also inhibits tumor growth. For instance, anti-VEGF antibodies blocking VEGF activities may be combined with anti-ErbB antibodies (e.g. HERCEPTIN® anti-HER2 antibody) in a treatment of metastatic breast cancer. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies.

The antibody of the invention (and adjunct therapeutic agent) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, e.g. by injections, such as intravenous or subcutaneous injections, depending in part on whether the administration is brief or chronic.

The antibody composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as used hereinbefore or about from 1 to 99% of the heretofore employed dosages.

For the prevention or treatment of disease, the appropriate dosage of an antibody of the invention (when used alone or in combination with other agents such as chemotherapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody is an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is by itself or when combined with another compositions effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice, such as cancer. Moreover, the article of manufacture may comprise (a) a first container with a composition contained therein, wherein the composition comprises an antibody of the invention; and (b) a second container with a composition contained therein, wherein the composition comprises a further cytotoxic agent. The article of manufacture in this embodiment of the invention may further comprise a package insert indicating that the first and second antibody compositions can be used to treat a particular condition, e.g. cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following are examples of the methods and compositions of the invention. It is understood that various other embodiments may be practiced, given the general description provided above.

### EXAMPLES

This example describes construction and purification of bispecific antibodies having a variant hinge region lacking disulfide-forming cysteine residues (“hingeless”). Construction of bispecific antibodies having wild type hinge sequence is also described; these antibodies can be used to assess efficiency of obtaining various species of antibody complexes.

### Construction of Expression Vectors

All plasmids for the expression of full-length antibodies were based on a separate cistron system (Simmons et al., J. Immunol. Methods, 263:133-147 (2002)) which relied on separate phoA promoters (AP) (Kikuchi et al., Nucleic Acids Res., 9:5671-5678 (1981)) for the transcription of heavy and light chains, followed by the trp Shine-Dalgarno sequences for translation initiation (Yanofsky et al., Nucleic Acids Res., 9:6647-6668 (1981) and Chang et al., Gene, 55:189-196 (1987)). Additionally, the heat-stable enterotoxin II signal sequence (STII) (Picken et al., Infect. Immun., 42:269-275 (1983) and Lee et al., Infect. Immun., 42:264-268 (1983)) was used for periplasmic secretion of heavy and light chains. Fine control of translation for both chains was achieved with previously described STII signal sequence variants of measured relative translational strengths, which contain silent codon changes in the translation initiation region (TIR) (Simmons and Yansura, Nature Biotechnol., 14:629-634 (1996) and Simmons et al., J. Immunol. Methods, 263:133-147 (2002)). Finally, the λ_{τ0} transcriptional terminator (Schlosstissek and Grosse, Nucleic Acids Res., 15:3185 (1987)) was placed downstream of the coding sequences for both chains. All plasmids use the framework of a pBR322-based vector system (Sutcliffe, Cold Spring Harbor Symp. Quant. Biol., 43:77-90 (1978)).

(i) Plasmid p5A6.11.Knob.Hg—

Two intermediate plasmids were required to generate the desired p5A6.11.Knob.Hg— plasmid. The variable domain of the 5A6 (anti-Fcγ-RIIb) chimeric light chain was first transferred onto the pVG11.VNERK.Knob plasmid to generate the intermediate plasmid p5A6.1.L.VG.1.H.Knob. The variable domain of the 5A6 chimeric heavy chain was then transferred onto the p5A6.1.L.VG.1.H.Knob plasmid to generate the intermediate plasmid p5A6.11.Knob plasmid. The following describes the preparation of these intermediate plasmids p5A6.1.L.VG.1.HC.Knob and p5A6.11.Knob followed by the construction of p5A6.11.Knob.Hg—

p5A6.1.L.VG.1.H.Knob

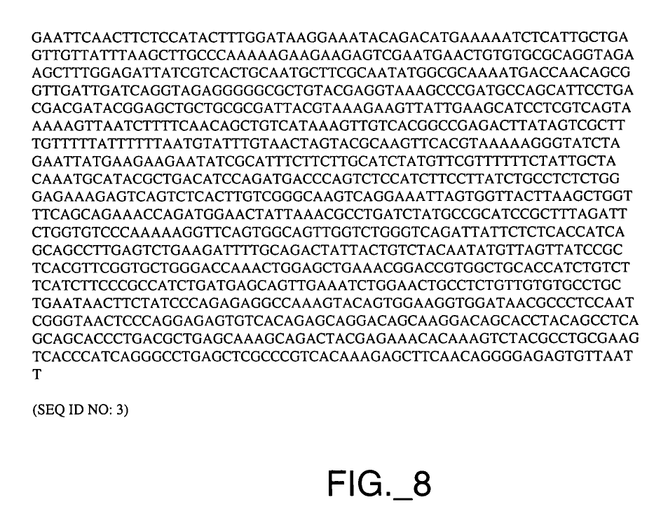

This plasmid was constructed in order to transfer the murine light variable domain of the 5A6 antibody to a plasmid compatible for generating the full-length antibody. The construction of this plasmid involved the ligation of two DNA fragments. The first was the pVG11.VNERK.Knob vector in which the small EcoRI-PacI fragment had been removed. The plasmid pVG11.VNERK.Knob is a derivative of the separate cistron vector with relative TIR strengths of 1-light and 1-heavy (Simmons et al., J. Immunol. Methods, 263:133-147 (2002)) in which the light and heavy variable domains have been changed to an anti-VEGF antibody (VNERK) with the “knob” mutation (T366W) (Merchant et al., Nature Biotechnology, 16:677-681 (1998)) and all the control elements described above. The second part of the ligation involved ligation of the sequence depicted in FIG. 8 into the EcoRI-PacI digested pVG11.VNERK.Knob vector described above. The sequence encodes the alkaline phosphatase promoter (phoA), STII signal sequence and the entire (variable and constant domains) light chain of the 5A6 antibody.

p5A6.11.Knob

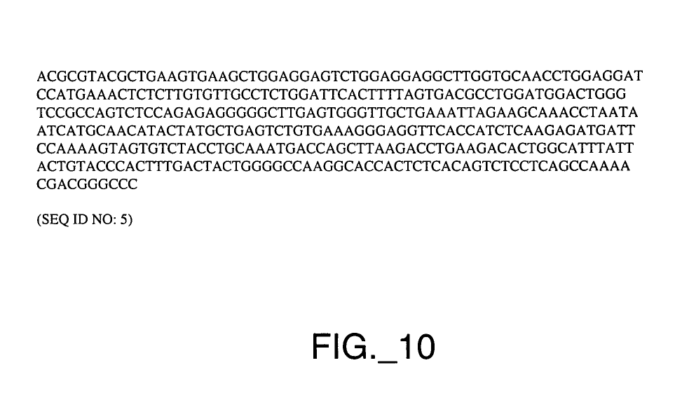

This plasmid was constructed to introduce the murine heavy variable domain of the 5A6 antibody into a human heavy chain framework to generate the chimeric full-length antibody. The construction of p5A6.11.Knob involved the ligation of two DNA fragments. The first was the p5A6.1.L.VG.1.H.Knob vector in which the small MluI-PspOMI fragment had been removed. The second part of the ligation involved ligation of the sequence depicted in FIG. 10 into the MluI-PspOMI digested p5A6.1.L.VG.1.H.Knob vector. The sequence encodes the last 3 amino acids of the STII signal sequence and approximately 119 amino acids of the murine heavy variable domain of the 5A6 antibody.

p5A6.11.Knob.Hg—

The p5A6.11.Knob.Hg— plasmid was constructed to express the full-length chimeric 5A6 hingeless Knob antibody. The construction of the plasmid involved the ligation of two DNA fragments. The first was the p5A6.11.Knob vector in which the small PspOMI-SacII fragment had been removed. The second part of the ligation was an approximately 514 base-pair PspOMI-SacII fragment from p4D5.22.Hg— encoding for approximately 171 amino acids of the human heavy chain in which the two hinge cysteines have been converted to serines (C226S, C229S, EU numbering scheme of Kabat, E. A., et al. (eds.) 1991, page 671 in Sequences of proteins of immunological interest, 5^{th} ed. Vol. 1., NIH, Bethesda, Md.). The plasmid p4D5.22.Hg— is a derivative of the separate cistron vector with relative TIR strengths of 2-light and 2-heavy (Simmons et al., J. Immunol. Methods, 263:133-147 (2002)) in which the light and heavy variable domains have been changed to an anti-HER2 antibody and the two hinge cysteines have been converted to serines (C226S, C229S).

(ii) Plasmid p5A6.22.Knob.Hg—

One intermediate plasmid was required to generate the desired p5A6.22.Knob.Hg— plasmid. The phoA promoter and the STII signal sequence—relative TIR strength of 2 for light chain were first transferred onto the p5A6.11.Knob.Hg— plasmid to generate the intermediate plasmid p5A6.21.Knob.Hg—. The following describes the preparation of the intermediate plasmid p5A6.21.Knob.Hg— followed by the construction of p5A6.22.Knob.Hg—

p5A6.21.Knob.Hg—

This plasmid was constructed to introduce the STII signal sequence—relative TIR strength of 2 for the light chain. The construction of p5A6.21.Knob.Hg— involved the ligation of three DNA fragments. The first was the p5A6.11.Knob.Hg— vector in which the small EcoRI-PacI fragment had been removed. The second part of the ligation was an approximately 658 base-pair NsiI-PacI fragment from the p5A6.11.Knob.Hg— plasmid encoding the light chain for the chimeric 5A6 antibody. The third part of the ligation was an approximately 489 base-pair EcoRI-NsiI PCR fragment generated from the p1H1.22.Hg—, using the following primers:

(SEQ ID NO: 1)

5′-AAAGGGAAAGAATTCAACTTCTCCAGACTTTGGATAAGG

(SEQ ID NO: 2)

5′-AAAGGGAAAATGCATTTGTAGCAATAGAAAAAACGAA

The plasmid p1H1.22.Hg— is a derivative of the separate cistron vector with relative TIR strengths of 2-light and 2-heavy (Simmons et al., J. Immunol. Methods, 263:133-147 (2002)) in which the light and heavy variable domains have been changed to a rat anti-Tissue Factor antibody in which the two hinge cysteines have been converted to serines (C226S, C229S).

p5A6.22.Knob.Hg—

This plasmid was constructed to introduce the STII signal sequence—with a relative TIR strength of 2 for the heavy chain. The construction of p5A6.22.Knob involved the ligation of two DNA fragments. The first was the p5A6.21.Knob.Hg— vector in which the small PacI-MluI fragment had been removed. The second part of the ligation was an approximately 503 base-pair PacI-MluI fragment from the p1H1.22.Hg— plasmid encoding the λ_{τ0} transcriptional terminator for the light chain, the phoA promoter, and the STII signal sequence—relative TIR strength of 2 for the heavy chain.

(iii) Plasmid p22E7.11.Hole.Hg—

Two intermediate plasmids were required to generate the desired p22E7.11.Hole.Hg— plasmid. The variable domain of the 22E7 (anti-IgE-R) chimeric light chain was first transferred onto the pVG11.VNERK.Hole plasmid to generate the intermediate plasmid p22E7.1.L.VG.1.H.Hole. The variable domain of the 22E7 chimeric heavy chain was then transferred onto the p22E7.1.L.VG.1.H.Hole plasmid to generate the intermediate plasmid p22E7.11.Hole plasmid. The following describes the preparation of these intermediate plasmids p22E7.1.L.VG.1.H.Hole and p22E7.11.Hole followed by the construction of p22E7.11.Hole.Hg—

p22E7.1.L.VG.1.H.Hole

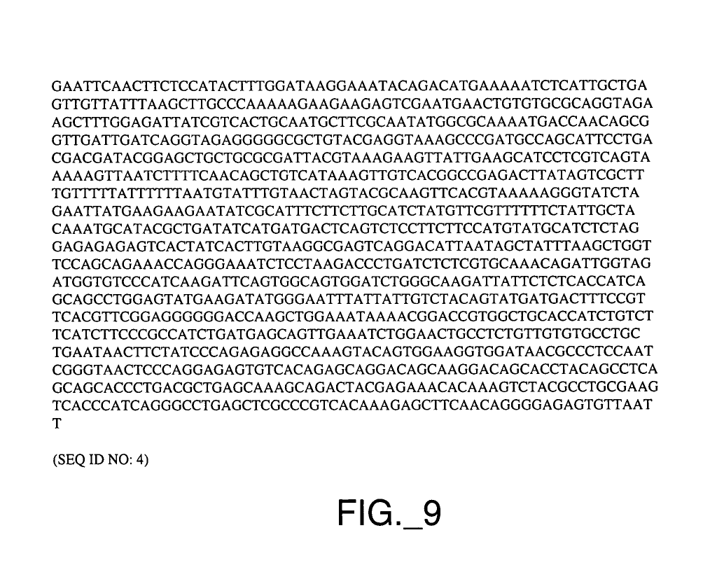

This plasmid was constructed in order to transfer the murine light variable domain of the 22E7 antibody to a plasmid compatible for generating the full-length antibody. The construction of this plasmid involved the ligation of two DNA fragments. The first was the pVG11.VNERK.Hole vector in which the small EcoRI-PacI fragment had been removed. The plasmid pVG11.VNERK.Hole is a derivative of the separate cistron vector with relative TIR strengths of 1-light and 1-heavy (Simmons et al., J. Immunol. Methods, 263:133-147 (2002)) in which the light and heavy variable domains have been changed to an anti-VEGF antibody (VNERK) with the “hole” mutations (T366S, L368A, Y407V) (Merchant et al., Nature Biotechnology, 16:677-681 (1998)) and all the control elements described above. The second part of the ligation involved ligation of the sequence depicted in FIG. 9 into the EcoRI-PacI digested pVG11.VNERK.Hole vector described above. The sequence encodes the alkaline phosphatase promoter (phoA), STII signal sequence and the entire (variable and constant domains) light chain of the 22E7 antibody.

p22E7.11.Hole

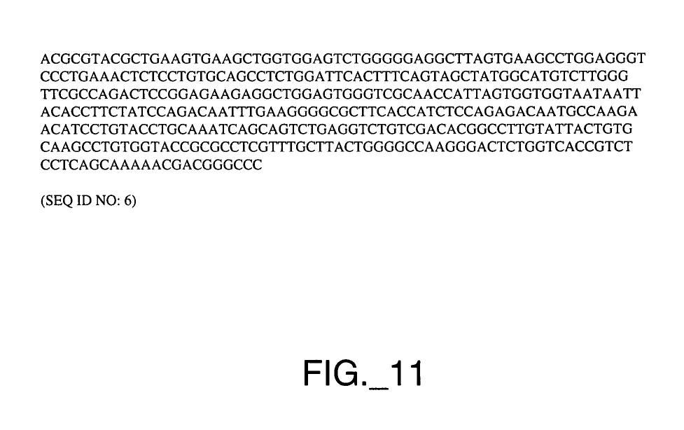

This plasmid was constructed to introduce the murine heavy variable domain of the 22E7 antibody into a human heavy chain framework to generate the chimeric full-length antibody. The construction of p22E7.11.Knob involved the ligation of two DNA fragments. The first was the p22E7.1.L.VG.1.H.Hole vector in which the small MluI-PspOMI fragment had been removed. The second part of the ligation involved ligation of the sequence depicted in FIG. 11 into the MluI-PspOMI digested p22.E7.1.L.VG.1.H.Hole vector. The sequence encodes the last 3 amino acids of the STII signal sequence and approximately 123 amino acids of the murine heavy variable domain of the 22E7 antibody.

p22E7.11.Hole.Hg—

The p22E7.11.Hole.Hg— plasmid was constructed to express the full-length chimeric 22E7 hingeless Hole antibody. The construction of the plasmid involved the ligation of two DNA fragments. The first was the p22E7.11.Hole vector in which the small PspOMI-SacII fragment had been removed. The second part of the ligation was an approximately 514 base-pair PspOMI-SacII fragment from p4D5.22.Hg— encoding for approximately 171 amino acids of the human heavy chain in which the two hinge cysteines have been converted to serines (C226S, C229S).

(iv) Plasmid p22E7.22.Hole.Hg—

One intermediate plasmid was required to generate the desired p22E7.22.Hole.Hg— plasmid. The phoA promoter and the STII signal sequence—relative TIR strength of 2 for light chain were first transferred onto the p22E7.11.Hole.Hg— plasmid to generate the intermediate plasmid p22E7.21.Hole.Hg—. The following describes the preparation of the intermediate plasmid p22E7.21.Hole.Hg— followed by the construction of p22E7.22.Hole.Hg—

p22E7.21.Hole.Hg—

This plasmid was constructed to introduce the STII signal sequence—with a relative TIR strength of 2 for the light chain. The construction of p22E7.21.Hole.Hg— involved the ligation of three DNA fragments. The first was the p22E7.11.Hole.Hg— vector in which the small EcoRI-PacI fragment had been removed. The second part of the ligation was an approximately 647 base-pair EcoRV-PacI fragment from the p22E7.11.Hole.Hg— plasmid encoding the light chain for the chimeric 22E7 antibody. The third part of the ligation was an approximately 500 base-pair EcoRI-EcoRV fragment from the p1H1.22.Hg— plasmid encoding the alkaline phosphatase promoter (phoA) and STII signal sequence.

p22E7.22.Hole.Hg—

This plasmid was constructed to introduce the STII signal sequence—with a relative TIR strength of 2 for the heavy chain. The construction of p22E7.22.Hole.Hg— involved the ligation of three DNA fragments. The first was the p22E7.21.Hole.Hg— vector in which the small EcoRI-MluI fragment had been removed. The second part of the ligation was an approximately 1141 base-pair EcoRI-PacI fragment from the p22E7.21.Hole.Hg— plasmid encoding the alkaline phosphatase promoter, STII signal sequence, and the light chain for the chimeric 22E7 antibody. The third part of the ligation was an approximately 503 base-pair PacI-MluI fragment from the p1H1.22.Hg— plasmid encoding the λ_{τ0} transcriptional terminator for the light chain, the phoA promoter, and the STII signal sequence—with a relative TIR strength of 2 for the heavy chain.

### Antibody Expression—5A6 Knob and 22E7 Hole

The “knobs into holes” technology was used to promote heterodimerization to generate full length bispecific anti-FcγRIIb (5A6) and anti-IgE-R (22E7) antibody. The “knobs into holes” mutations in the CH3 domain of Fc sequence has been reported to greatly reduce the formation of homodimers (Merchant et al., Nature Biotechnology, 16:677-681 (1998)). Constructs were prepared for the anti-FcγRIIb component (p5A6.11.Knob) by introducing the “knob” mutation (T366W) into the Fc region, and the anti-IgE-R component (p22E7.11.Hole) by introducing the “hole” mutations (T366S, L368A, Y407V).

Small-scale inductions of the antibodies were carried out using the plasmids p5A6.11.Knob for knob anti-FcγRIIb and p22E7.11.Hole for hole anti-IgE-R. Each plasmid possessed relative TIR strengths of 1 for both light and heavy chains. For small scale expression of each construct, the E. coli strain 33D3 (W3110 ΔfhuA (ΔtonA) ptr3 lac Iq lacL8 ΔompT Δ(nmpc-fepE) degP41 kan^{R}) was used as host cells. Following transformation, selected transformant picks were inoculated into 5 mL Luria-Bertani medium supplemented with carbenicillin (50 μg/mL) and grown at 30° C. on a culture wheel overnight. Each culture was then diluted (1:100) into C.R.A.P. phosphate-limiting media (Simmons et al., J. Immunol. Methods 263:133-147 (2002)). Carbenicillin was then added to the induction culture at a concentration of 50 μg/mL and the culture was grown for approximately 24 hours at 30° C. on a culture wheel. Unless otherwise noted, all shake flask inductions were performed in a 5 mL volume.

Non-reduced whole cell lysates from induced cultures were prepared as follows: (1) 1 OD₆₀₀-mL induction samples were centrifuged in a microfuge tube; (2) each pellet was resuspended in 90 μL TE (10 mM Tris pH 7.6, 1 mM EDTA); (3) 10 μL of 100 mM iodoacetic acid (Sigma I-2512) was added to each sample to block any free cysteines and prevent disulfide shuffling; (4) 20 μL of 10% SDS was added to each sample. The samples were vortexed, heated to about 90° C. for 3 minutes and then vortexed again. After the samples had cooled to room temperature, 750 μL acetone was added to precipitate the protein. The samples were vortexed and left at room temperature for about 15 minutes. Following centrifugation for 5 minutes in a microcentrifuge, the supernatant of each sample was aspirated off, and each protein pellet was resuspended in 50 μL dH₂0+50 μL 2× NOVEX SDS sample buffer. The samples were then heated for 4 minutes at about 90° C., vortexed well and allowed to cool to room temperature. A final 5 minute centrifugation was then done and the supernatants were transferred to clean tubes.

Reduced whole cell lysates from induced cultures were prepared as follows: (1) 1 OD₆₀₀-mL induction samples were centrifuged in a microfuge tube; (2) each pellet was resuspended in 90 μL TE (10 mM Tris pH 7.6, 1 mM EDTA); (3) 10 μL of 1 M dithiothreitol (Sigma D-5545 ) was added to each sample to reduce disulfide bonds; (4) 20 μL of 10% SDS was added to each sample. The samples were vortexed, heated to about 90° C. for 3 minutes and then vortexed again. After the samples had cooled to room temperature, 750 μL acetone was added to precipitate the protein. The samples were vortexed and left at room temperature for about 15 minutes. Following centrifugation for 5 minutes in a microcentrifuge, the supernatant of each sample was aspirated off and each protein pellet was resuspended in 10 μL 1 M dithiothreitol+40 μL dH20+50 μL 2× NOVEX SDS sample buffer. The samples were then heated for 4 minutes at about 90° C., vortexed well and allowed to cool to room temperature. A final 5 minute centrifugation was then done and the supernatants were transferred to clean tubes.

Following preparation, 5-8 μL of each sample was loaded onto a 10 well, 1.0 mm NOVEX manufactured 12% Tris-Glycine SDS-PAGE and electrophoresed at ˜120 volts for 1.5-2 hours. The resulting gels were then either stained with Coomassie Blue or used for Western blot analysis.

For Western blot analysis, the SDS-PAGE gels were electroblotted onto a nitrocellulose membrane (NOVEX) in 10 mM CAPS buffer, pH 11+3% methanol. The membrane was then blocked using a solution of 1× NET (150 mM NaCl, 5 mM EDTA, 50 mM Tris pH 7.4, 0.05% Triton X-100)+0.5% gelatin for approximately 30 min−1 hours rocking at room temperature. Following the blocking step, the membrane was placed in a solution of 1× NET+0.5% gelatin+anti-Fab antibody (peroxidase-conjugated goat IgG fraction to human IgG Fab; CAPPEL #55223) for an anti-Fab Western blot analysis. The anti-Fab antibody dilution ranged from 1:50,000 to 1:1,000,000 depending on the lot of antibody. Alternatively, the membrane was placed in a solution of 1× NET+0.5% gelatin+anti-Fc antibody (peroxidase-conjugated goat IgG fraction to human Fc fragment; BETHYL #A80-104P-41) for an anti-Fc Western blot analysis. The anti-Fc antibody dilution ranged from 1:50,000 to 1:250,000 depending on the lot of the antibody. The membrane in each case was left in the antibody solution overnight at room temperature with rocking. The next morning, the membrane was washed a minimum of 3×10 minutes in 1× NET+0.5% gelatin and then 1×15 minutes in TBS (20 mM Tris pH 7.5, 500 mM NaCl). The protein bands bound by the anti-Fab antibody and the anti-Fc antibody were visualized using Amersham Pharmacia Biotech ECL detection and exposing the membrane to X-Ray film.

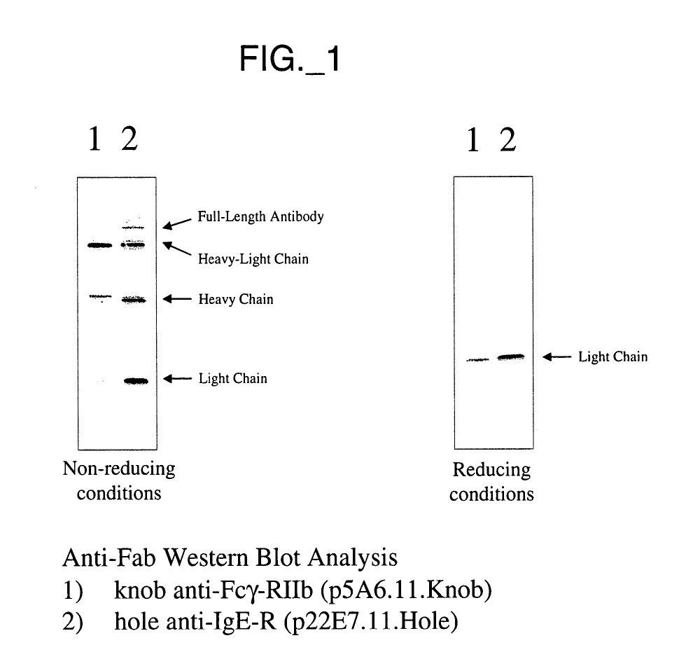

The anti-Fab Western blot results for the p5A6.11.Knob (knob anti-Fcγ-RIIb) and p22E7.11.Hole (hole anti-IgE-R) antibody expression are shown in FIG. 1. They reveal the expression of fully folded and assembled heavy-light (HL) chain species for the knob anti-Fcγ-RIIb antibody in lane 1 and the hole anti-IgE-R antibody in lane 2. It is important to note that the anti-Fab antibody has different affinities for different variable domains of the light chain. The anti-Fab antibody generally has a lower affinity for the heavy chain. For the non-reduced samples, the expression of each antibody results in the detection of the heavy-light chain species. Notably, the full-length antibody species is detectable for the hole anti-IgE-R antibody, however it is only a small proportion of total fully folded and assembled antibody species. The folding and assembly of the full-length antibody species is not favored as a result of the inclusion of the “knob” mutation for the anti-Fcγ-RIIb antibody and the “hole” mutations for the anti-IgE-R antibody. For the reduced samples, the light chain is detected for the knob anti-Fcγ-RIIb antibody and the hole anti-IgE-R antibody.

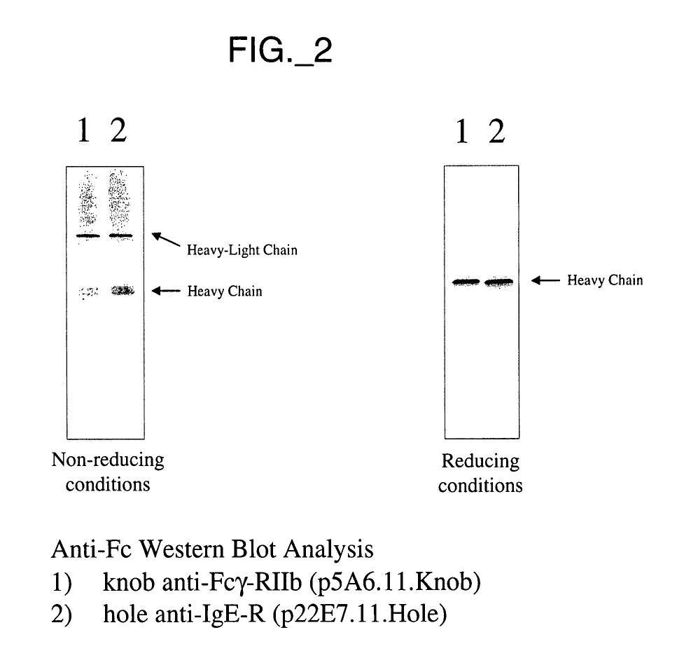

Similarly, the anti-Fc Western blot results are shown in FIG. 2 and they also reveal the expression of fully folded and assembled heavy-light (HL) chain species for the knob anti-Fcγ-RIIb antibody in lane 1 and the hole anti-IgE-R antibody in lane 2. The anti-Fc antibody is not able to bind light chain, and therefore it is not detected. For the non-reduced samples, the expression of each antibody again results in the detection of the heavy-light chain species, but not the full-length antibody species. For the reduced samples, there are similar quantities of heavy chain detected for the knob anti-Fcγ-RIIb antibody and the hole anti-IgE-R antibody.

### Expression of 5A6 Knob Hinge Variant and 22E7 Hole Hinge Variant Antibodies

The primary antibody species with the p5A6.11.Knob and p22E7.11.Hole constructs were the fully folded and assembled heavy-light (HL) chain species. However, in order to facilitate the method of preparation herein described for the bispecific anti-Fc□RIIb/anti-IgE-R (5A6/22E7) antibody, the hinge sequence of the two heavy chains were modified by substituting the two hinge cysteines with serines (C226S, C229S, EU numbering scheme of Kabat, E. A. et al. (eds.) 1991. page 671 in Sequences of proteins of immunological interest, 5th ed. Vol. 1. NIH, Bethesda Md.). Hinge variants are also referred to below as “hingeless”.

Constructs were prepared for the knob anti-Fcγ-RIIb (5A6) antibody and the hole anti-IgE-R (22E7) antibody comprising hinge variants having C226S, C229S substitutions. Two constructs were prepared for each antibody. One construct had a relative TIR strength of 1 for both light and heavy chains and the second construct had a relative TIR strength of 2 for both light and heavy chains.

The knob anti-Fcγ-RIIb antibody (p5A6.11.Knob), the hole anti-IgE-R antibody (p22E7.11.Hole), the knob hingeless anti-Fcγ-RIIb antibodies (p5A6.11.Knob.Hg— & p5A6.22.Knob.Hg—), and the hole hingeless anti-IgE-R antibodies (p22E7.11.Hole.Hg— & p22E7.22.Hole.Hg—) were then expressed in the same manner as described above. Whole cell lysates were prepared, separated by SDS-PAGE, transferred to nitrocellulose, and detected with the goat anti-human Fab conjugated antibody and goat anti-human Fc conjugated antibody described above.

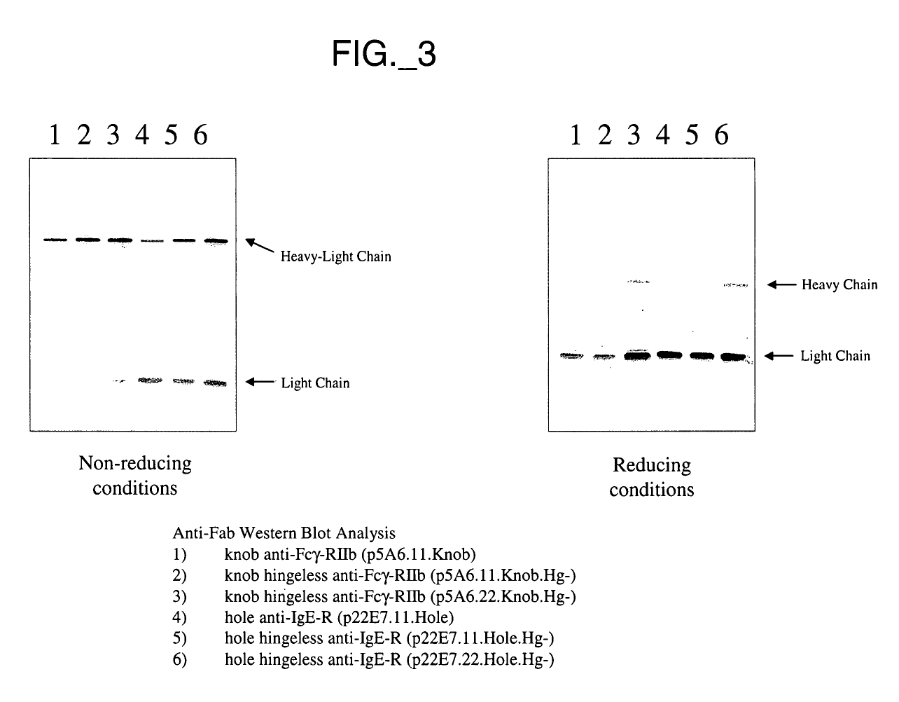

The anti-Fab Western blot results are shown in FIG. 3 and they show a significant improvement in folding and assembly of the heavy-light (HL) chain species for the knob hingeless anti-Fcγ-RIIb antibody (relative TIR strengths—1 for light chain and 1 for heavy chain) in lane 2 and the hole hingeless anti-IgE-R antibody (relative TIR strengths—1 for light chain and 1 for heavy chain) in lane 5. In addition, the anti-Fab Western blot results show an increase in the folding and assembly of the heavy-light (HL) chain species for the knob hingeless anti-Fcγ-RIIb antibody (lane 3) and the hole hingeless anti-IgE-R antibody (lane 6) when the relative TIR strengths for light and heavy chain are increased from 1 to 2. Again, it is important to note that the anti-Fab antibody has different affinities for different variable domains of the light chain and generally has a lower affinity for the heavy chain. For the non-reduced samples, the expression of each antibody results in the detection of the heavy-light chain species, but not the full-length antibody species as a result of the conversion of the hinge cysteines to serines. There are significant improvements in the folding and assembly of the heavy-light (HL) chain species for both the knob hingeless anti-Fcγ-RIIb and hole hingeless anti-IgE-R antibodies when the two hinge cysteines are converted to serines and again when the relative TIR strengths for light and heavy chains are increased from 1 to 2. For the reduced samples, the heavy and light chains are detected for the different anti-Fcγ-RIIb and anti-IgE-R antibodies. The increase in the quantities of heavy and light chains is detected when the relative TIR strengths are increased from 1 to 2.

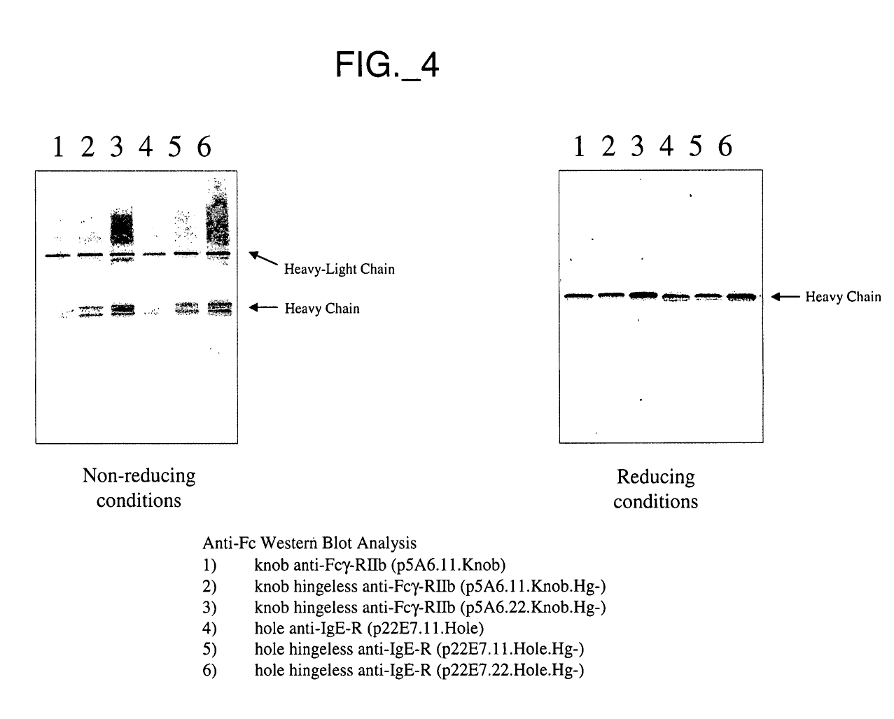

Similarly, the anti-Fc Western blot results in FIG. 4 show significant improvement in the folding and assembly of the heavy-light (HL) chain species for both the knob hingeless anti-Fcγ-RIIb and hole hingeless anti-IgE-R antibodies when the two hinge cysteines are converted to serines and again when the relative TIR strengths for light and heavy chains are increased from 1 to 2. The anti-Fc antibody is not able to bind light chain, and therefore it is not detected. For the reduced samples, the heavy chain is detected for the different anti-Fcγ-RIIb and anti-IgE-R antibodies. The increase in the quantities of heavy chains is detected when the relative TIR strengths are increased from 1 to 2.

Ease and efficiency of obtaining purified and functional bispecific antibodies was further assessed in the context of antibodies having a variant hinge region as described above.

### Purification of Bispecifc Antibody Components

1. Extraction from E. coli Paste

Frozen E. coli paste was thawed and suspended in 5 volumes (v/w) distilled water, adjusted to pH 5 with HCl, centrifuged, and the supernatant discarded. The insoluble pellet was resuspended in 5-10 volumes of a buffer at pH 9 using a polytron (Brinkman), and the supernatant retained following centrifugation. This step was repeated once.

The insoluble pellet was then resuspended in 5-10 volumes of the same buffer, and the cells disrupted by passage through a microfluidizer (Microfluidics). The supernatant was retained following centrifugation.

The supernatants were evaluated by SDS polyacrylamide gel electrophoresis (SDS-PAGE) and Western blots, and those containing the single-armed antibody (ie: a band corresponding to the molecular weight of a single heavy chain plus light chain) were pooled.

2. Protein-A Affinity Chromatography

The pooled supernatants were adjusted to pH8, and ProSep-A beads (Millipore) were added (approximately 250 ml beads per 10 liters). The mixture was stirred for 24-72 hours at 4° C., the beads allowed to settle, and the supernatant poured off. The beads were transferred to a chromatography column (Amersham Biosciences XK50), and washed with 10 mM tris buffer pH7.5. The column was then eluted using a pH gradient in 50 mM citrate, 0.1M NaCl buffer. The starting buffer was adjusted to pH6, and the gradient formed by linear diluton with pH2 buffer.

Fractions were adjusted to pH5 and 2M urea by addition of 8M urea and tris base, then evaluated by SDS-PAGE and pooled.

3. Cation Exchange Chromatography

An S-Sepharose Fast Flow column (Amersham Biosciences) was equilibrated with 2M urea, 25 mM MES pH5.5. The ProSep-A eluate pool was diluted with an equal volume of equilibration buffer, and loaded onto the column. After washing with equilibration buffer, then with 25 mM MES pH5.5, the column was developed with a linear gradient of 0-1M NaCl in 25 mM MES, pH5.5. Fractions were pooled based on SDS-PAGE analysis.

4. Hydrophobic Interaction Chromatography

A HI-Propyl column (J. T. Baker) was equilibrated with 0.5M sodium sulfate, 25 mM MES pH6. The S-Fast Flow eluate was adjusted to 0.5M Sodium sulfate, pH6, loaded onto the column, and the column developed with a gradient of 0.5-0M sodium sulfate in 25 mM MES, pH6. Fractions were pooled based on SDS-PAGE analysis.

5. Size Exclusion Chromatography

The HI-Propyl eluate pool was concentrated using a CentriPrep YM10 concentrator(Amicon), and loaded onto a Superdex SX200 column (Amersham Biosciences) equilibrated with 10 mM succinate or 10 mM histidine in 0.1M NaCl, pH6, and the column developed at 2.5 ml/m. Fractions were pooled based on SDS-PAGE.

### Annealing of Antibody Components to Generate Bispecific Antibodies

Two similar (but not identical) annealing methods are described below, both of which resulted in good yields of bi-specific antibodies. Heavy chains of the antibodies and antibody components described below contain a variant hinge region as described above.

Annealing Hinge Variant 5A6Knob and Hinge Variant 22E7Hole—Version 1

Purified 5A6Knob and 22E7Hole antibodies in 25 mM MES pH5.5, 0.5 M NaCl, were mixed in equal molar ratios based on their concentrations. The mixture was then heated at 50° C. for 5 minutes to 1 hour. This annealing temperature was derived from the melting curves previously described for these CH3 variants (Atwell, S., et al. J. Mol. Biol. 270:26-35, 1997). The annealed antibody was then subjected to analysis to determine its bispecificity.

Analysis of Bispecificity

1) Isoelectric Focusing

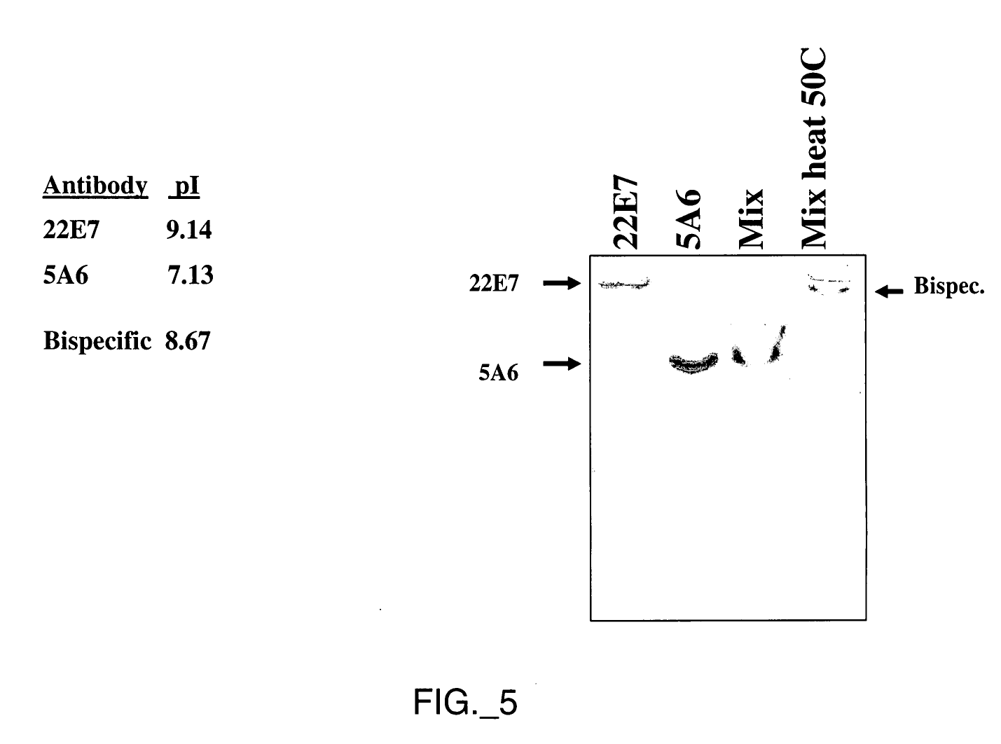

The easiest way to verify that the annealed antibody was truly bispecific was to apply samples for isoelectric focusing analysis. The 5A6Knob antibody has a pI of 7.13 while the 22E7Hole has a pI of 9.14. The bispecific 5A6Knob/22E7Hole antibody has a pI of 8.67. FIG. 5 shows the movement of the 5A6Knob, 22E7Hole and bispecific 5A6Knob/22E7Hole (before and after heating) antibodies on an isoelectric focusing gel (Invitrogen, Novex pH3-10 IEF) after staining with Coomassie Blue. While there is some annealing upon mixing at room temperature, the heating to 50° C. appears to promote completion of the process. The appearance of a new protein band with a pI in between that of 5A6Knob and 22E7Hole verifies the formation of the bispecific antibody.

2) Affinity Column Analysis

The behaviors of the 5A6Knob, 22E7Hole, and bispecific 5A7Knob/22E7Hole antibodies were observed on FcγRIIb affinity columns. A human FcγRIIb (extracellular domain)-GST fusion protein was coupled to a solid support in a small column according to the manufacturer's instructions (Pierce, UltraLink Immobilization Kit #46500). 5A6Knob, 22E7Hole, and bispecific 5A6Knob/22E7Hole antibodies in PBS (137 mM NaCl, 2.7 mM KCl, 8 mM Na₂HPO₄, 1.5mM KH₂PO₄, pH 7.2) were loaded onto three separate FcγRIIb affinity columns at approximately 10-20% of the theoretical binding capacity of each column. The columns were then washed with 16 column volumes of PBS. The column flow-throughs for the loading and wash were collected, combined, and concentrated approximately 10-fold in Centricon Microconcentrators (Amicon). Each concentrate in the same volume was then diluted 2 fold with 2× SDS sample buffer and analyzed by SDS-PAGE (Invitrogen, Novex Tris-Glycine). The protein bands were transferred to nitrocellulose by electroblotting in 20 mM Na₂HPO₄ pH 6.5, and probed with an anti-human IgG Fab peroxidase conjugated antibody (CAPPELL#55223). The antibody bands were then detected using Amersham Pharmacia Biotech ECL according to the manufacturer's instructions.

The results of this analysis are shown in FIG. 6. The FcγRIIb affinity column should retain the 5A6Knob antibody and the 5A6Knob/22E7Hole bispecific antibody if it is truly bispecific. The 22E7Hole antibody should flow through as is shown in the figure. The lack of any antibody detected in the 5A6Knob/22E7Hole bispecific lane suggests that it is truly bispecific.

Annealing Hinge Variant 5A6Knob and Hinge Variant 22E7Hole—Version 2

The antibody components (single arm 5A6Knob and 22E7Hole) were purified as described above.

The ‘heterodimer’ was formed by annealing at 50° C., using a slight molar excess of 5A6, then purified on a cation exchange column.

5A6(Knob) 5 mg and 22E7(Hole) 4.5 mg were combined in a total volume of 10 ml 8 mM succinate, 80 mM NaCl buffer, adjusted to 20 mM tris, pH7.5.

The mixture was heated to 50° C. in a water bath for 10 minutes, then cooled to 4° C.

Analysis of Bispecificity

1. Isoelectric Focusing

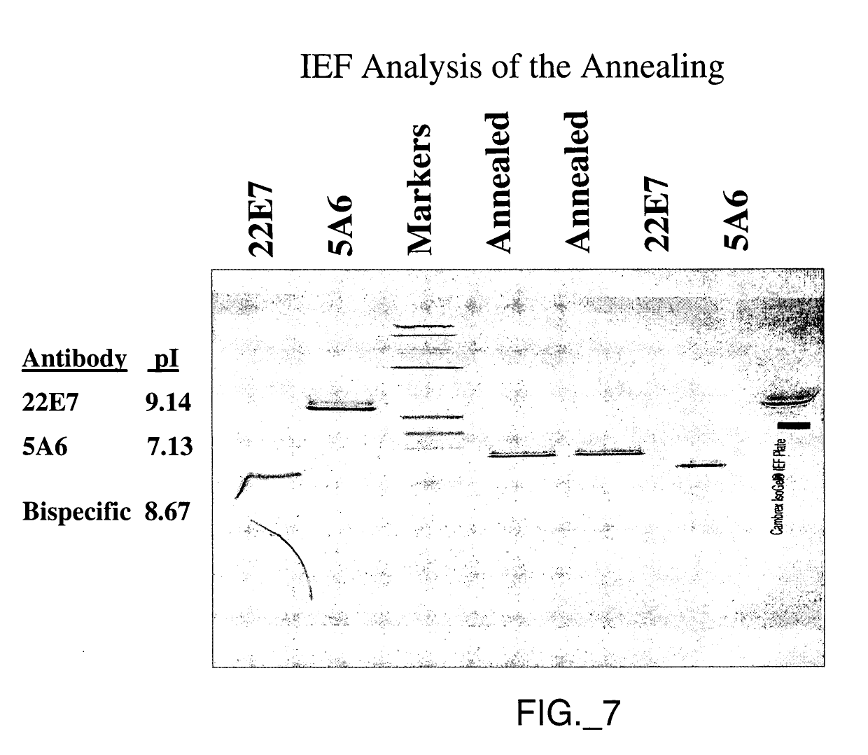

Analysis on an isoelectric focusing gel (Cambrex, pH7-11) showed formation of a single band at pI˜8.5 in the annealing mixture, corresponding to bispecific antibody (which has a calculcated pI of 8.67). See FIG. 7.

2. Purification On a Cation Exchange Column

A 5 ml CM-Fast Flow column (HiTrap, Amersham Biosciences) was equilibrated with a buffer at pH5.5 (30 mM MES, 20 mM hepes, 20 mM imidazole, 20 mM tris, 25 mM NaCl). The annealed pool was diluted with an equal volume of equilibration buffer and adjusted to pH5.5, loaded onto the column, and washed with equilibration buffer. The column was developed at 1 ml/min with a gradient of pH5.5 to pH9.0 in the same buffer, over 30 minutes.

Fractions were analyzed by IEF, which revealed that 5A6 was eluted ahead of the heterodimer. Analysis by light scattering of the pooled fractions containing heterodimer revealed no monomer.

1. A method of making a bispecific antibody comprising a first heavy chain polypeptide paired with a first light chain polypeptide and a second heavy chain polypeptide paired with a second light chain polypeptide, wherein the first heavy chain polypeptide and the second heavy chain polypeptide each comprises a variant hinge region incapable of inter-heavy chain disulfide linkage, said method comprising:.
(a) expressing the first heavy chain polypeptide and the first light chain polypeptide in a first host cell;
(b) expressing the second heavy chain polypeptide and the second light chain polypeptide in a second host cell;
(c) isolating the heavy and light chain polypeptides of (a) and (b);
(d) annealing the isolated polypeptides of (c) to form a bispecific antibody comprising a first arm comprising the first heavy chain paired with the first light chain and a second arm comprising the second heavy chain paired with the second light chain.

2. A method comprising:
(a) expressing in a first host cell a first pair of immunoglobulin heavy and light chain polypeptides that are capable of forming a first target molecule binding arm,
(b) expressing in a second host cell a second pair of immunoglobulin heavy and light chain polypeptides that are capable of forming a second target molecule binding arm,
wherein heavy chain polypeptides of the first pair and second pair comprise a variant hinge region incapable of inter-heavy chain disulfide linkage, and wherein light chains of the first pair and second pair comprise different variable domain sequences,
(c) isolating the polypeptides from the host cells of step (a),
(d) contacting the polypeptides in vitro under conditions permitting multimerization of the isolated polypeptides to form a substantially homogeneous population of antibodies having binding specificity to two distinct target molecules.

3. A method comprising:
(a) obtaining a sample comprising a mixture of 4 polypeptides, wherein the 4 polypeptides are a first pair of immunoglobulin heavy and light chain polypeptides that are capable of forming a first target molecule binding arm, and a second pair of immunoglobulin heavy and light chain polypeptides that are capable of forming a second target molecule binding arm, wherein heavy chain polypeptides of the first pair and second pair comprise a variant hinge region incapable of inter-heavy chain disulfide linkage,
(b) incubating the 4 polypeptides under conditions permitting multimerization of the polypeptides to form a substantially homogeneous population of antibodies having binding specificity to two distinct target molecules.

4. A method comprising:
incubating 4 immunoglobulin polypeptides under conditions permitting multimerization of the polypeptides to form a substantially homogeneous population of antibodies, wherein each antibody has binding specificity to two distinct target molecules,
wherein the 4 immunoglobulin polypeptides are a first pair of immunoglobulin heavy and light chain polypeptides that are capable of forming a first target molecule binding arm, and a second pair of immunoglobulin heavy and light chain polypeptides that are capable of forming a second target molecule binding arm,
wherein each heavy chain polypeptide of the first pair and second pair comprises a variant hinge region incapable of inter-heavy chain disulfide linkage.

5. A method comprising:
incubating a first pair of immunoglobulin heavy and light chain polypeptides, and a second pair of immunoglobulin heavy and light chain polypeptides, under conditions permitting multimerization of the first and second pair of polypeptides to form a substantially homogeneous population of antibodies,
wherein the first pair of polypeptides is capable of binding a first target molecule;
wherein the second pair of polypeptides is capable of binding a second target molecule;
wherein each heavy chain polypeptide of the first pair and second pair comprises a variant hinge region incapable of inter-heavy chain disulfide linkage.

6. A method comprising:
incubating a first pair of immunoglobulin heavy and light chain polypeptides, and a second pair of immunoglobulin heavy and light chain polypeptides, under conditions permitting multimerization of the first and second pair of polypeptides to form a substantially homogeneous population of antibodies,
wherein the first pair of polypeptides is capable of binding a first target molecule;
wherein the second pair of polypeptides is capable of binding a second target molecule;
wherein Fc polypeptide of the first heavy chain polypeptide and Fc polypeptide of the second heavy chain polypeptide meet at an interface, and the interface of the second Fc polypeptide comprises a protuberance which is positionable in a cavity in the interface of the first Fc polypeptide.

7. The method of claim 1 wherein each heavy chain polypeptide of the first pair and second pair comprises a variant hinge region incapable of inter-heavy chain disulfide linkage.

8. The method of claim 1 wherein the first pair and second pair of immunoglobulin heavy and light chain polypeptides are obtained from separate expression units.

9. The method of claim 8 wherein an expression unit is a cell.

10. The method of claim 8 wherein an expression unit is a cell culture.

11. The method of claim 8 wherein an expression unit is an in vitro protein expression sample/system.

12. The method of claim 1 wherein said inter-heavy chain disulfide linkage is between Fc regions.

13. The method of claim 1 wherein said variant heavy chain hinge region lacks a cysteine residue capable of forming a disulfide linkage.

14. The method of claim 1 wherein said disulfide linkage is intermolecular.

15. The method of claim 1 wherein said intermolecular disulfide linkage is between cysteines of two immunoglobulin heavy chains.

16. The method of claim 1 wherein a hinge region cysteine residue that is normally capable of forming a disulfide linkage is deleted.

17. The method of claim 1 wherein a hinge region cysteine residue that is normally capable of forming a disulfide linkage is substituted with another amino acid.

18. The method of claim 1 wherein said cysteine residue is substituted with serine.

19. The method of claim 1, wherein said antibody comprises a heavy chain constant domain and a light chain constant domain.

20. The method of claim 1 wherein the heavy chains comprise at least a portion of a human CH2 and/or CH3 domain.

21. The method of claim 1 wherein one or both pairs of heavy and light chain polypeptides are humanized.

22. The method of claim 1 wherein said antibody is humanized.

23. The method of claim 1 wherein the antibody is a full-length antibody.

24. The method of claim 23 wherein said full-length antibody comprises a heavy chain and a light chain.

25. The method of claim 1 wherein one or both pairs of heavy and light chain polypeptides are human.

26. The method of claim 1 wherein said antibody is human.

27. The method of claim 1 wherein the antibody is an antibody fragment comprising at least a portion of human CH2 and/or CH3 domain.

28. The method of claim 27 wherein said antibody fragment is an Fc fusion polypeptide.

29. The method of claim 1 wherein the antibody is selected from the group consisting of IgG, IgA and IgD.

30. The method of claim 1 wherein the antibody is IgG.

31. The method of claim 1 wherein the antibody is IgG1.

32. The method of claim 1 wherein the antibody is IgG2.

33. The method of claim 1 wherein the antibody is a therapeutic antibody.

34. The method of claim 1 wherein the antibody is an agonist antibody.

35. The method of claim 1 wherein the antibody is an antagonistic antibody.

36. The method of claim 1 wherein the antibody is a diagnostic antibody.

37. The method of claim 1 wherein the antibody is a blocking antibody.

38. The method of claim 1 wherein the antibody is a neutralizing antibody.

39. The method of claim 1 wherein the antibody is capable of binding to a tumor antigen.

40. The method of claim 39 wherein the tumor antigen is not a cell surface molecule.

41. The method of claim 39 wherein the tumor antigen is not a cluster differentiation factor.

42-47. (canceled)

48. The method of claim 1 wherein light chains of the first pair and second pair comprise different variable domain sequences.

49. The method of claim 1 wherein Fc polypeptide of the first heavy chain polypeptide and Fc polypeptide of the second heavy chain polypeptide meet at an interface, and the interface of the second Fc polypeptide comprises a protuberance which is positionable in a cavity in the interface of the first Fc polypeptide.

50. The method of claim 49 wherein at least 90% of the polypeptides form said bispecific antibody.

51. The method of claim 49 wherein the second Fc polypeptide has been altered from a template/original polypeptide to encode the protuberance or the first Fc polypeptide has been altered from a template/original polypeptide to encode the cavity, or both.

52. The method of claim 49 wherein the second Fc polypeptide has been altered from a template/original polypeptide to encode the protuberance and the first Fc polypeptide has been altered from a template/original polypeptide to encode the cavity, or both.

53. The method of claim 49 wherein the first Fc polypeptide and the second Fc polypeptide meet at an interface, wherein the interface of the second Fc polypeptide comprises a protuberance which is positionable in a cavity in the interface of the first Fc polypeptide, and wherein the cavity or protuberance, or both, have been introduced into the interface of the first and second Fc polypeptides respectively.

54. The method of claim 1, wherein said bispecific antibody is capable of specifically binding two target molecules.

55. The method of claim 1, wherein the first arm specifically binds a first target molecule and the second arm specifically binds a second target molecule.

56. The method of claim 1 wherein the first host cell and the second host cell are in separate cell cultures.

57. The method of claim 1 wherein the first host cell and the second host cell are in a mixed culture comprising both host cells.

58. The method of claim 1 wherein the host cells are prokaryotic.

59. The method of claim 58 wherein the prokaryotic host cell is E. coli.

60. The method of claim 59, wherein the E. coli is of a strain deficient in endogenous protease activities.

61. The method of claim 1, wherein said host cell is eukaryotic.

62. The method of claim 61, wherein the host cell is CHO.

63. The method of claim 1, wherein nucleic acids encoding the polypeptides are operably linked to translational initiation regions (TIRs) of approximately equal strength.

64. The method of claim 1 wherein wherein the annealing or contacting step comprises incubating the mixture of isolated polypeptides at room temperature.

65. The method of claim 1 wherein wherein the annealing or contacting step comprises heating the mixture of isolated polypeptides.

66. The method of claim 65 wherein the mixture is heated to at least 40° C.

67. The method of claim 65 wherein the mixture is heated to at least 50° C.

68. The method of claim 65 wherein the mixture is heated to between about 40° C. and 60° C.

69. The method of claim 65 wherein the mixture is at 50° C.

70. The method of claim 1 wherein the annealing or contacting step comprises heating the mixture of isolated polypeptides for at least 2 minutes.

71. The method of claim 65 wherein the mixture is cooled after heating.

72. The method of claim 1 wherein the annealing or contacting step comprises incubating the mixture of isolated polypeptides at a pH at or between about 4 to about 11.

73. The method of claim 72 wherein the pH is about 5.5.

74. The method of claim 72 wherein the pH is about 7.5.

75. The method of claim 1 wherein the annealing or contacting step comprises incubating the mixture of isolated polypeptides in a denaturant.

76. The method of claim 75 wherein the denaturant is urea.

77. The method of claim 1 wherein the annealing or contacting step does not include chemical conjugation between the first and second heavy chain polypeptides.

78. The method of claim 1 wherein at least 75% of the polypeptides are in a complex comprising the first heavy and light chain pair and the second heavy and light pair.

79. The method of claim 1 wherein no more than 10% of the isolated polypeptides are present as monomers or dimers prior to the step of purifying the antibodies.

80. The method of claim 1 wherein light chains of the first pair and second pair comprise different variable domain sequences.

81. The method of claim 1 wherein the first and second heavy-light chain pairs each comprises heavy and light chains disulfide linked to each other.

82. The method of claim 1 wherein the first pair and the second pair of polypeptides are provided in approximately equimolar amount [ratio] in the annealing or contacting step;

83. The method of claim 1 wherein difference in pI values between the first pair and second pair is at least 0.5.

84. A bispecific antibody produced according to the method of claim 1.

85. A bispecific antibody comprising a first pair of heavy and light chain polypeptides, and a second pair of heavy chain and light chain polypeptides, wherein the light chain polypeptides comprise different variable domain sequences, and wherein the heavy chains comprise a variant hinge region incapable of inter-heavy chain disulfide linkage.

86. An isolated nucleic acid encoding the antibody of claim 84.

87. A host cell comprising the nucleic acid of claim 86.

88. The host cell of claim 87 wherein nucleic acid encoding each pair of heavy and light chain polypeptides is present in a single vector.

89. The host cell of claim 87 wherein nucleic acid encoding heavy chain and light chain polypeptide of each pair is present in separate vectors.

90. A composition comprising one or more recombinant nucleic acids which collectively encode the bispecific antibody of claim 84.

91. A composition comprising a bispecific antibody of claim 84 and a carrier.

92. A composition comprising a population of immunoglobulins wherein at least 80% of the immunoglobulins is a bispecific antibody of claim 84.


SEQUENCE LISTING (6 sequences)

SEQ ID NO: 1 - DNA, 39 bp, Artificial sequence
  Sequence is synthesized
aaagggaaagaattcaacttctccagactttggataagg

SEQ ID NO: 2 - DNA, 37 bp, Artificial sequence
  Sequence is synthesized
aaagggaaaatgcatttgtagcaatagaaaaaacgaa

SEQ ID NO: 3 - DNA, 1141 bp, Artificial sequence
  Sequence is synthesized
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagtacgcaagttcacgtaaaaagggtatctagaattatgaagaagaatatcgcatttcttcttgcatctatgttcgttttttctattgctacaaatgcatacgctgacatccagatgacccagtctccatcttccttatctgcctctctgggagaaagagtcagtctcacttgtcgggcaagtcaggaaattagtggttacttaagctggtttcagcagaaaccagatggaactattaaacgcctgatctatgccgcatccgctttagattctggtgtcccaaaaaggttcagtggcagttggtctgggtcagattattctctcaccatcagcagccttgagtctgaagattttgcagactattactgtctacaatatgttagttatccgctcacgttcggtgctgggaccaaactggagctgaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatt

SEQ ID NO: 4 - DNA, 1141 bp, Artificial sequence
  Sequence is synthesized
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagtacgcaagttcacgtaaaaagggtatctagaattatgaagaagaatatcgcatttcttcttgcatctatgttcgttttttctattgctacaaatgcatacgctgatatcatgatgactcagtctccttcttccatgtatgcatctctaggagagagagtcactatcacttgtaaggcgagtcaggacattaatagctatttaagctggttccagcagaaaccagggaaatctcctaagaccctgatctctcgtgcaaacagattggtagatggtgtcccatcaagattcagtggcagtggatctgggcaagattattctctcaccatcagcagcctggagtatgaagatatgggaatttattattgtctacagtatgatgactttccgttcacgttcggaggggggaccaagctggaaataaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatt

SEQ ID NO: 5 - DNA, 370 bp, Artificial sequence
  Sequence is synthesized
acgcgtacgctgaagtgaagctggaggagtctggaggaggcttggtgcaacctggaggatccatgaaactctcttgtgttgcctctggattcacttttagtgacgcctggatggactgggtccgccagtctccagagagggggcttgagtgggttgctgaaattagaagcaaacctaataatcatgcaacatactatgctgagtctgtgaaagggaggttcaccatctcaagagatgattccaaaagtagtgtctacctgcaaatgaccagcttaagacctgaagacactggcatttattactgtacccactttgactactggggccaaggcaccactctcacagtctcctcagccaaaacgacgggccc

SEQ ID NO: 6 - DNA, 382 bp, Artificial sequence
  Sequence is synthesized
acgcgtacgctgaagtgaagctggtggagtctgggggaggcttagtgaagcctggagggtccctgaaactctcctgtgcagcctctggattcactttcagtagctatggcatgtcttgggttcgccagactccggagaagaggctggagtgggtcgcaaccattagtggtggtaataattacaccttctatccagacaatttgaaggggcgcttcaccatctccagagacaatgccaagaacatcctgtacctgcaaatcagcagtctgaggtctgtcgacacggccttgtattactgtgcaagcctgtggtaccgcgcctcgtttgcttactggggccaagggactctggtcaccgtctcctcagcaaaaacgacgggccc